(19) United States
(12) Patent Application Publication
(10) Pub. No.: US 2026/0262953 A1
(43) Pub. Date: Sep. 10, 2026

(54) METHOD AND SYSTEM TO NON-INVASIVELY ASSESS ELEVATED PULMONARY CAPILLARY WEDGE PRESSURE

(71) Applicant: Analytics for Life Inc., Toronto (CA)

(72) Inventors: Navid Nemati, Toronto (CA); Timothy William Fawcett Burton, Ottawa (CA); Farhad Fathieh, North York (CA); Horace R. Gillins, Toronto (CA); Ian Shadforth, Toronto (CA); Shyam Ramchandani, Kingston (CA); Charles R. Bridges, Auburndale, MA (US)

(21) Appl. No.: 19/560,585

(22) Filed: Mar. 9, 2026

Related U.S. Application Data
(60) Provisional application No. 63/768,628, filed on Mar. 7, 2025.

(51) Int. Cl. A61B 5/021; A61B 5/00; A61B 5/35

(52) U.S. Cl. CPC A61B 5/02116; A61B 5/35; A61B 5/7221; A61B 5/7225

(57) ABSTRACT

A clinical evaluation system and associated method for non-invasive assessment of pulmonary capillary wedge pressure in a subject. that utilize one or more biophysical signals acquired non-invasively from a subject to derive features or parameters that are processed by a computational model or classifier, including a machine-learned classifier. The model is configured to estimate metrics associated with the physiological state of the subject, including an estimated PCWP or the presence or absence of elevated PCWP, which serves as an indicator of elevated left ventricular filling pressure and may be used to assess disease states such as heart failure with or without preserved ejection fraction. In this manner, the disclosed system enables non-invasive estimation of PCWP, thereby providing clinically relevant information regarding LV filling pressures without requiring invasive catheter-based measurement.

### RELATED APPLICATIONS

This U.S. application claims priority to, and the benefit of, U.S. Provisional Patent Application No. 63/768,628, filed Mar. 7, 2025, entitled “Method and System to Non-Invasively Assess Pulmonary Capillary Wedge Pressure,” which is incorporated by reference herein in its entirety.

### FIELD OF THE INVENTIONS

The present disclosure generally relates to methods and systems, and its development system and environment, that can be used to assess for the presence, non-presence, severity, and/or localization of pathologies or indication of one, utilizing biophysical measurements, including in the estimation of the presence of elevated or abnormal pulmonary capillary wedge pressure (PCWP) and/or left-ventricular end-diastolic pressure (LVEDP) using cardiac and photoplethysmographic-related measurements.

### BACKGROUND

There are numerous methods and systems for assisting a healthcare professional in diagnosing disease. Some of these involve the use of invasive or minimally invasive techniques, radiation, exercise or stress, or the use of pharmacological agents, sometimes in combination, with their attendant risks and other disadvantages.

Pulmonary capillary wedge pressure (PCWP) provides an indirect estimate of left atrial pressure (LAP), which is often difficult in having to place a catheter in a right atrium and punch through the interatrial septum, which can cause damage to the septum and provide potential harm to the patient. Pulmonary wedge pressure is also referred to as pulmonary arterial wedge pressure, pulmonary artery occlusion pressure, or cross-sectional pressure, is the pressure measured by wedging a pulmonary artery catheter with an inflated balloon into a small pulmonary arterial branch. For example, a balloon-tipped, multi-lumen catheter may be inserted into a peripheral vein (e.g., jugular or femoral vein) and then advanced into the right atrium, right ventricle, pulmonary artery, and then into a branch of the pulmonary artery.

PCWP measurements can be used to diagnose the severity of left ventricular failure and to quantify the degree of mitral valve stenosis, either of which can elevate LAP and, therefore, PCWP. PCWP is also important to measure when evaluating pulmonary hypertension.

It is desirable to have a system that can assist healthcare professionals in the diagnosis of cardiac disease and various other diseases and conditions without the aforementioned disadvantages.

### SUMMARY

A clinical evaluation system and associated method are disclosed for non-invasive assessment of pulmonary capillary wedge pressure (PCWP) in a subject. In clinical practice, PCWP obtained through right-heart catheterization is used as a surrogate measure for estimating left ventricular end-diastolic pressure (LVEDP) and for assessing left-sided cardiac filling pressures. However, such measurements require invasive catheterization procedures. The present disclosure provides systems and methods that utilize one or more biophysical signals acquired non-invasively from a subject to derive features or parameters that are processed by a computational model or classifier, including a machine-learned classifier. The model is configured to estimate metrics associated with the physiological state of the subject, including an estimated PCWP or the presence or absence of elevated PCWP, which serves as an indicator of elevated left ventricular filling pressure and may be used to assess disease states such as heart failure with or without preserved ejection fraction. In this manner, the disclosed system enables non-invasive estimation of PCWP, thereby providing clinically relevant information regarding LV filling pressures without requiring invasive catheter-based measurement.

In another aspect, a clinical evaluation system and method are disclosed that facilitate the use of features or parameters extracted from biophysical signals in a model or classifier (e.g., a machine-learned classifier) to estimate metrics associated with the physiological state of a patient, including for the presence or non-presence of elevated pulmonary capillary wedge pressure (PCWP), as an example indicator of a disease medical condition that could be assessed by using the system and method described herein. The biophysical signals are acquired, in preferred embodiments, non-invasively from surface sensors placed on a patient while the patient is at rest. The estimated metric may be used to assist a physician or other healthcare providers in diagnosing the presence, non-presence, severity, and/or localization of diseases or conditions or in the treatment of the diseases or conditions.

The estimation or determined likelihood of the presence or non-presence of elevated PCWP can supplant, augment, or replace other evaluation or measurement modalities for the assessment of elevated PCWP. In some cases, a determination can take the form of a numerical score and related information.

A development system and environment are disclosed to develop the machine-learned models or classifiers of the clinical evaluation system. The feature sets, analytical and modeling framework of the development system and environment, and configuration of the clinical evaluation system can be applied, in parts and/or in combination with other features and model techniques, to the assessment or estimation of metrics associated with other pathologies or indications of one.

As used herein, the term “feature” (in the context of machine learning and pattern recognition and as used herein) generally refers to an individual measurable property or characteristic of a phenomenon being observed. A feature is defined by analysis and may be determined in groups in combination with other features from a common model or analytical framework.

As used herein, the term “metric” refers to an estimation or likelihood of the presence, non-presence, severity, and/or localization (where applicable) of one or more diseases, conditions, or indications of either, in a physiological system or systems. Notably, the exemplified methods and systems can be used in certain embodiments described herein to acquire biophysical signals and/or to otherwise collect data from a patient and to evaluate those signals and/or data in signal processing and classifier operations to evaluate for a disease, condition, or indicator of one that can supplant, augment, or replace other evaluation modalities via one or more metrics. In some cases, a metric can take the form of a numerical score and related information.

In the context of cardiovascular and respiratory systems, examples of diseases and conditions to which such metrics can relate include, for example: (i) heart failure (e.g., left-side or right-side heart failure; heart failure with preserved ejection fraction (HFpEF)), (ii) HF with preserved EF (HFpEF) and HF with reduced EF (HFrEF), (iii) various forms of pulmonary hypertension (PH) including without limitation pulmonary arterial hypertension (PAH), or other subtypes to which PCWP can assist with subtyping of PH (iv) abnormal left ventricular ejection fraction (LVEF), and various other diseases or conditions, (v) cardiac ischemia, as a elevated LVEDP can be indicative of ischemia. An example indicator of certain forms of heart failure is the presence or non-presence of elevated or abnormal pulmonary capillary wedge pressure (PCWP). PCWP assists with subtyping PH—different subtypes have very different treatment strategies.

As discussed above, the exemplified methods and systems facilitate analyses and modeling framework that can accurately and precisely estimate the presence or non-presence of elevated left-ventricular end-diastolic pressure using only non-invasive measurements.

In an aspect, a method is disclosed to non-invasively estimate presence of elevated PCWP in a mammalian subject, the method comprising obtaining, by one or more processors, a first biophysical signal data set associated with a first photoplethysmographic signal and a second photoplethysmographic signal, wherein the first biophysical data set has been acquired over multiple cardiac cycles of the subject; obtaining, by the one or more processors, a second biophysical signal data set associated with a cardiac signal, wherein the second biophysical data set has been simultaneously acquired with the first biophysical signal data set over the multiple cardiac cycles; determining, by the one or more processors utilizing at least a portion of the first and second biophysical signal data sets, a plurality of values associated with a plurality of features; and determining, by the one or more processors, an estimated value for presence of elevated PCWP using the plurality of values associated with the plurality of features, wherein the estimated value (e.g., as an elevated PCWP or HF score) for the presence of abnormal pulmonary capillary wedge pressure is outputted for use in a diagnosis of expected left heart failure (LHF) or to direct treatment of the expected left heart failure.

In some embodiments, the plurality of features include a feature set selected from the group consisting of one or more depolarization or repolarization wave propagation associated features; one or more depolarization wave propagation deviation associated features; one or more cycle variability associated features; one or more dynamical system associated features; one or more cardiac waveform topologic and variations associated features (e.g., linear features); one or more photoplethysmogram (PPG) waveform topologic and variations associated features (e.g., PPG linear features); one or more cardiac or PPG signal power spectral density associated features (e.g., cardiac/PPG visual features); one or more cardiac or PPG signal visual associated features; and one or more predictability features.

In some embodiments, the one or more depolarization or repolarization wave propagation associated features are configured to quantify a propagative characteristic (e.g., velocity, trajectory, orbital frequency (3D rotation rate), and/or planarity of the wave) of a ventricular depolarization (VD) wave or a ventricular repolarization (VR) wave defined in the cardiac signal in three-dimensional space.

In some embodiments, the one or more depolarization or repolarization wave propagation associated features are configured to quantify a propagative characteristic (e.g., velocity, trajectory, orbital frequency (3D rotation rate), and/or planarity of the wave) of a bandpass filtered ventricular depolarization (VD) wave or a ventricular repolarization (VR) wave defined in the cardiac signal in three-dimensional space (e.g., a QRS_max_Curvature_sub5 feature, which calculates a maximum curvature for the VD wave at a 5th frequency sub-band; a T_max_OrbitalFrequency_sub14 feature, which calculates the maximum orbital frequency for the VR wave at the 14th frequency sub-band).

In some embodiments, the one or more depolarization wave propagation deviation associated features are configured to quantify deviations, via evaluation of high-frequency and low-amplitude patterns, of a VD wave trajectory from a trajectory of a three-dimensional modeled VD wave, wherein the three-dimensional modeled VD wave is a representative VD wave below about 40 Hz.

In some embodiments, the one or more cycle variability associated features are configured to quantify beat-to-beat variations of the cardiac signal in comparison of each respective beat to a determined template beat.

In some embodiments, the one or more dynamical system features (e.g., cardiac and PPG dynamical system analysis features) are configured to quantify dynamical characteristics (e.g., Lyapunov exponent, correlation dimension, entropy, mutual information, correlation, and nonlinear filtering) of the second biophysical data set (e.g., a LEX feature, which is a Lyapunov exponent of a biopotential channel orth1).

In some embodiments, the one or more cardiac waveform topologic and variations associated features (e.g., cardiac linear analysis features) are configured to quantify both cardiac measurements and variations (e.g., waveform amplitudes, durations, heart rate, and morphologies) of the second biophysical signal data set.

In some embodiments, the one or more PPG waveform topologic and variations associated features (e.g., PPG linear analysis features) are configured to quantify both PPG measurements and variations (e.g., peak amplitudes, peak-to-peak distances, angles between points, and various ratios of the first photoplethysmographic signal and/or a second photoplethysmographic signal, a velocity photoplethysmogram generated from the first photoplethysmographic signal and/or a second photoplethysmographic signal, and an acceleration photoplethysmogram generated from the first photoplethysmographic signal and/or a second photoplethysmographic signal) of the first biophysical signal data set.

In some embodiments, the one or more cardiac or PPG signal power spectral density associated features are configured to quantify a power spectrum and frequency content (e.g., power spectrum and coherence) of the first photoplethysmographic signal and a second photoplethysmographic signal.

In some embodiments, the one or more cardiac or PPG signal power spectral density associated features are configured to quantify a power spectrum and frequency content (e.g., power spectrum, cross-spectral analysis, coherence) of the second biophysical signal data set.

In some embodiments, the one or more cardiac or PPG visual associated features are configured to quantify geometric parameters of a three-dimensional phase space generated from the first or second biophysical data set.

In some embodiments, the one or more predictability features are configured to quantify a predictability measure of the signal (e.g., predictability error and statistics of residues condensed from nonlinear filtering techniques), wherein the predictability measure measures an ability of a model trained on 75% of a signal to predict the remaining 25% (e.g., a StdResX feature, which is a standard deviation of residue from channel orth1).

In some embodiments, the one or more processors are located in a cloud platform.

In some embodiments, the one or more processors are located in a local computing device.

In some embodiments, the method further includes determining, by the one or more processors, via an outlier detection analysis of the first biophysical signal data set and the second biophysical signal data set, presence of outliers, wherein the outlier detection analysis comprises a machine learned module configured to detect arrhythmia (i.e., sudden changes in heart rates) and/or incorrect lead configurations in the mammalian subject.

In some embodiments, the machine learned module is further configured to detect an anomalous waveform that is missing or has extra waveform elements (e.g., presence and/or absence of any of the depolarization and/or repolarization waves) in the first photoplethysmographic signal, the second photoplethysmographic signal, and/or the cardiac signal.

In some embodiments, the method further includes preprocessing, by the one or more processors, the second biophysical signal data set, wherein the preprocessing includes i) transient time removal operation, DC offset removal operation, and baseline wander removal operation.

In some embodiments, the method further includes determining, by the one or more processors, via signal quality assessment analysis of the first biophysical signal data set, a plurality of photoplethysmographic noise score of the first biophysical signal data set, including i) a first photoplethysmographic noise score associated with rapid changes in the first or second photoplethysmographic signals and ii) a second photoplethysmographic noise score associated with measurement saturation.

In some embodiments, the method further includes determining, by the one or more processors, via signal quality assessment analysis of the second biophysical signal data set, a plurality of biopotential noise score of the second biophysical signal data set, including a first biopotential noise score associated with biopotential powerline interference and a second biopotential noise score associated with bipotential high frequency noise.

In some embodiments, the step of determining the estimated value for presence of abnormal PCWP values is determined by determining, by the one or more processors, a plurality of values for the plurality of features in two or more models, including a model selected from the group consisting of a linear model (e.g., Elastic Net), a decision tree model (XGB Classifier), a random forest model, a support vector machine model, a neural network model, and wherein the two or more models are combined (e.g., averaged) in an ensemble model that outputs the estimated value for the presence of abnormal PCWP.

In some embodiments, the ensemble model includes at least the two or more models and a model (e.g., a sigmoid model) associated with a body mass index (BMI) value of the mammalian subject.

In some embodiments, the plurality of features are selected from a pool of candidate features by a selection module.

In some embodiments, the selection module comprises a univariate feature assessment analysis that evaluates data sets (e.g., of a plurality of data sets) defined with respect to a positive pathology label associated with an elevated PCWP and a negative pathology label associated with a normal PCWP.

In some embodiments, the data sets are defined by a positive and negative pathology label (e.g., acquired from left heart catheterization (LHC)) selected from the group consisting of a PCWP value greater than or equal to 18 mmHg and a PCWP value less than or equal to 18 mmHg.

In some embodiments, the univariate feature assessment analysis is configured to determine a receiver-operating characteristic curve, a t-test, or a normalized mutual information analysis for presence of the elevated pulmonary capillary wedge pressure.

In some embodiments, the method further includes removing a feature from a plurality of candidate features having a high occurrence of extraction errors (e.g., prior to the univariate feature assessment analysis).

In some embodiments, the selection module comprises a cross-validation analysis (e.g., a stratified k-fold cross-validation analysis, e.g., 10-fold cross-validation analysis) for a set of trained models, wherein a set of data set is randomly sampled (e.g., 80%) as a training data set and a remainder is used as a validation data set in a feature permutation importance analysis.

In some embodiments, the feature permutation importance analysis quantifies a utility of a feature in combination with all other assessed features.

In some embodiments, the plurality of features are configured in a machine-learned model.

In some embodiments, the machine-learned model is trained using hyperparameters selected from a group of hyperparameters that have been evaluated in a hyperparameter sensitivity analysis.

In another aspect, a method is disclosed to reject an acquired biophysical signal data set as an outlier signal during an analysis to non-invasively estimate presence of elevated PCWP in a mammalian subject, the method comprising obtaining, by one or more processors, a first biophysical signal data set associated with-a first photoplethysmographic signal and a second photoplethysmographic signal, wherein the first biophysical data set has been acquired over multiple cardiac cycles of the subject; obtaining, by the one or more processors, a second biophysical signal data set associated with a cardiac signal, wherein the second biophysical data set has been simultaneously acquired with the first biophysical signal data set over the multiple cardiac cycles; determining, by the one or more processors and/or remotely by one or more cloud-based services or systems, via an outlier detection analysis of the first biophysical signal data set and the second biophysical signal data set, presence of outliers, wherein the outlier detection analysis comprises a machine learned model (e.g., forest model) configured to at least one of i) sudden changes in heart rates in the mammalian subject or ii) an anomalous waveform that is missing or has extra waveform elements; and rejecting, by the one or more processors and/or remotely by one or more cloud-based services or systems, the first and second biophysical signal data sets based on the outlier detection analysis, wherein the rejection generates a notification to be presented at i) measurement equipment used to acquire the first or second biophysical signal data set or ii) a remote terminal.

In some embodiments, the method further includes generating, by the one or more processors, a notification of at least one of the first and second biophysical signal data set being an outlier dataset based on the determination, wherein a non-rejected first biophysical signal data set and the second biophysical signal data set are used to estimate one or more values associated with a presence of an expected disease state or condition, and wherein the value is subsequently outputted for use in a diagnosis of the expected disease state or condition or to direct treatment of the expected disease state or condition.

In some embodiments, the machine learned model is configured using an unsupervised learning algorithm (e.g., Isolation Forest) configured to evaluate randomly selected features of the plurality of features and to evaluate a selected feature using random splitting points between maximum and minimum values of a set of features.

In some embodiments, the plurality of features include a feature set selected from the group consisting of one or more depolarization or repolarization wave propagation associated features; one or more depolarization wave propagation deviation associated features; one or more cycle variability associated features; one or more dynamical system associated features; one or more cardiac waveform topologic and variations associated features (e.g., linear features); one or more PPG waveform topologic and variations associated features (e.g., PPG linear features); one or more cardiac or PPG signal power spectral density associated features (e.g., cardiac/PPG visual features); one or more cardiac or PPG signal visual associated features; and one or more predictability features.

In some embodiments, the method further includes determining, by the one or more processors and/or remotely by one or more cloud-based services or systems, via a signal quality or cycle variability analysis of the acquired first and second biophysical signal data sets; and rejecting, by the one or more processors and/or remotely by one or more cloud-based services or systems, the first and second biophysical signal data sets based on the signal quality or cycle variability analysis, wherein the rejection generates a second notification to be presented at i) measurement equipment used to acquire the first or second biophysical signal data set or ii) a remote terminal.

In another aspect, a method is disclosed to configure a model to non-invasively estimate presence of a metric associated with an abnormal condition or disease state, the method comprising selecting, via a feature selection operation (e.g., univariate feature selection), a plurality of features from a plurality of candidate feature sets; training the plurality of selected features in one or more models, to produce a plurality of candidate models, wherein the one or more models include a model selected from the group consisting of a linear model (e.g., Elastic Net), a decision tree model (XGB Classifier), a random forest model, a support vector machine model, a neural network model, and wherein each of the one or more models is configured to output an estimated value associated with a presence of an expected disease state or condition or condition, wherein the estimated value is subsequently outputted for use in a diagnosis of the expected disease state or condition or to direct treatment of the expected disease state or condition; performing a stratified k-fold cross-validation operation of the plurality of trained candidate models; and validating the cross-validated models.

In some embodiments, the feature selection operation includes a univariate feature assessment analysis that evaluates data sets (e.g., of a plurality of data sets) defined with respect to a positive pathology label associated with the expected disease state or condition and a negative pathology label associated with the expected disease state or condition.

In some embodiments, the method further includes performing a hyperparameter sensitivity analysis to determine stability values for hyperparameters of the plurality of trained candidate models; and removing a trained candidate model of the plurality of trained candidate models having determined unstable stability values.

In some embodiments, the data sets are defined by a positive and negative pathology label selected from the group consisting of a PCWP value greater than or equal to 18 mmHg and a PCWP value less than or equal to 18 mmHg.

In some embodiments, the data sets are defined by a positive and negative pathology label associated with the presence or non-presence of significant coronary arterial disease (CAD).

In some embodiments, the data sets are defined by a positive and negative pathology label associated with the presence or non-presence of pulmonary hypertension.

In some embodiments, the expected disease or condition can be diagnosed based on assessed indication and/or estimate of presence, non-presence, and/or severity of elevated or abnormal pulmonary capillary wedge pressure (PCWP).

In some embodiments, the expected disease state or condition includes left ventricular heart failure or left-sided heart failure.

In some embodiments, the expected disease state or condition includes coronary artery disease.

In some embodiments, the expected disease state or condition includes pulmonary hypertension.

In some embodiments, the expected disease state or condition includes pulmonary arterial hypertension.

In some embodiments, the expected disease state or condition includes pulmonary hypertension due to left heart disease.

In some embodiments, the expected disease state or condition includes rare disorders that lead to pulmonary hypertension.

In some embodiments, the expected disease state or condition includes right ventricular heart failure or right-sided heart failure.

In some embodiments, the expected disease state or condition includes systolic heart failure.

In some embodiments, the expected disease state or condition includes diastolic heart failure.

In some embodiments, the disease state or condition includes ischemic heart disease.

In some embodiments, the disease state or condition includes arrhythmia.

In another aspect, a system (e.g., cloud platform or local computing platform) is disclosed comprising one or more processors; and one or more memory having instructions respectively stored thereon, wherein execution of the instructions by the one or more processors cause the one or more processors to perform any one of the above-discussed method.

In another aspect, a non-transitory computer readable medium is disclosed comprising instructions stored thereon, wherein execution of the instructions by one or more processors cause the one or more processors to perform any one of the above-discussed method.

### BRIEF DESCRIPTION OF THE DRAWINGS

The accompanying drawings, which are incorporated in and constitute a part of this specification, illustrate embodiments and, together with the description, serve to explain the principles of the methods and systems.

Embodiments of the present invention may be better understood from the following detailed description when read in conjunction with the accompanying drawings. Such embodiments, which are for illustrative purposes only, depict novel and non-obvious aspects of the invention. The drawings include the following figures:

FIG. 1 shows a schematic diagram of the operation and workflow of an analytical engine or analyzer of a clinical evaluation system that can non-invasively estimate or determine a likelihood of the presence, non-presence, severity, and/or localization of a disease state, medical condition, or an indication of either in a patient.

FIG. 2 shows an example biophysical signal capture system or component and its use in non-invasively collecting biophysical signals of a patient in a clinical setting in accordance with an illustrative embodiment.

FIG. 3A shows an example method to estimate or determine the likelihood of elevated PCWP from non-invasively acquired biophysical signals.

FIG. 3B shows an example of sets of feature modules that can be used in the PCWP assessment operation and workflow.

FIG. 4 shows an example visualization output of the clinical evaluation system of FIG. 1.

FIG. 5 shows the performance of the clinical evaluation system of FIG. 1 with a ROC curve.

FIG. 6 is a diagram of a development system and environment that can be used to develop and assess a clinical evaluation system that can predict and/or estimate presence, non-presence, localization, and/or severity of a metric/estimate associated with a disease, condition, or indicator of one in a patient, including the clinical evaluation system of FIG. 1.

FIGS. 7A-7C shows aspects of the operation of the modules of the development system and environment of FIG. 6.

FIG. 8 shows a schematic diagram of an example implementation of the clinical evaluation system of FIG. 1.

### DETAILED DESCRIPTION

Each and every feature described herein, and each and every combination of two or more of such features, is included within the scope of the present invention provided that the features included in such a combination are not mutually inconsistent.

While the present disclosure is directed to the practical assessment of biophysical signals, e.g., raw or pre-processed photoplethysmographic signals, biopotential/cardiac signals, etc., in the diagnosis, tracking, and treatment of cardiac-related pathologies and conditions, such assessment can be applied to the diagnosis, tracking, and treatment (including without limitation surgical, minimally invasive, lifestyle, nutritional, and/or pharmacologic treatment, etc.) of any pathologies or conditions in which a biophysical signal is involved in any relevant system of a living body. The assessment may be used in the controls of medical equipment or wearable devices or in monitoring applications (e.g., to report respiration rate or associated waveforms generated using the biophysical signals as disclosed therein).

The terms “subject” and “patient” as used herein are generally used interchangeably to refer to those who had undergone analysis performed by the exemplary systems and methods.

The term “cardiac signal” as used herein refers to one or more signals directly or indirectly associated with the structure, function, and/or activity of the cardiovascular system—including aspects of that signal's electrical/electrochemical conduction—that, e.g., cause contraction of the myocardium. A cardiac signal may include, in some embodiments, biopotential signals or electrocardiogramaignals, e.g., those acquired via an electrocardiogram (ECG), the cardiac and photoplethysmographic waveform or signal capture or recording instrument later described herein, or other modalities.

The term “biophysical signal” as used herein includes but is not limited to one or more cardiac signal(s), neurological signal(s), ballistocardiographic signal(s), and/or photoplethysmographic signal(s), but it also encompasses more broadly any physiological signal from which information may be obtained. Not intending to be limited by example, one may classify biophysical signals into types or categories that can include, for example, electrical (e.g., certain cardiac and neurological system-related signals that can be observed, identified, and/or quantified by techniques such as the measurement of voltage/potential (e.g., biopotential), impedance, resistivity, conductivity, current, etc. in various domains such as time and/or frequency), magnetic, electromagnetic, optical (e.g., signals that can be observed, identified and/or quantified by techniques such as reflectance, interferometry, spectroscopy, absorbance, transmissivity, visual observation, photoplethysmography, and the like), acoustic, chemical, mechanical (e.g., signals related to fluid flow, pressure, motion, vibration, displacement, strain), thermal, and electrochemical (e.g., signals that can be correlated to the presence of certain analytes, such as glucose). Biophysical signals may in some cases be described in the context of a physiological system (e.g., respiratory, circulatory (cardiovascular, pulmonary), nervous, lymphatic, endocrine, digestive, excretory, muscular, skeletal, renal/urinary/excretory, immune, integumentary/exocrine and reproductive systems), one or more organ system(s) (e.g., signals that may be unique to the heart and lungs as they work together), or in the context of tissue (e.g., muscle, fat, nerves, connective tissue, bone), cells, organelles, molecules (e.g., water, proteins, fats, carbohydrates, gases, free radicals, inorganic ions, minerals, acids, and other compounds, elements, and their subatomic components. Unless stated otherwise, the term “biophysical signal acquisition” generally refers to any passive or active means of acquiring a biophysical signal from a physiological system, such as a mammalian or non-mammalian organism. Passive and active biophysical signal acquisition generally refers to the observation of natural or induced electrical, magnetic, optical, and/or acoustics emittance of the body tissue. Non-limiting examples of passive and active biophysical signal acquisition means include, e.g., voltage/potential, current, magnetic, optical, acoustic, and other non-active ways of observing the natural emittance of the body tissue, and in some instances, inducing such emittance. Non-limiting examples of passive and active biophysical signal acquisition means include, e.g., ultrasound, radio waves, microwaves, infrared and/or visible light (e.g., for use in pulse oximetry or photoplethysmography), visible light, ultraviolet light, and other ways of actively interrogating the body tissue that does not involve ionizing energy or radiation (e.g., X-ray). An active biophysical signal acquisition may involve excitation-emission spectroscopy (including, for example, excitation-emission fluorescence). The active biophysical signal acquisition may also involve transmitting ionizing energy or radiation (e.g., X-ray) (also referred to as “ionizing biophysical signal”) to the body tissue. Passive and active biophysical signal acquisition means can be performed in conjunction with invasive procedures (e.g., via surgery or invasive radiologic intervention protocols) or non-invasively (e.g., via imaging, ablation, heart contraction regulation (e.g., via pacemakers), catheterization, etc.).

The term “photoplethysmographic signal” as used herein refers to one or more signals or waveforms acquired from optical sensors that correspond to measured changes in light absorption by oxygenated and deoxygenated hemoglobin, such as light having wavelengths in the red and infrared spectra. Photoplethysmographic signal(s), in some embodiments, include a raw signal(s) acquired via a pulse oximeter or a photoplethysmogram (PPG). In some embodiments, photoplethysmographic signal(s) are acquired from off-the-shelf, custom, and/or dedicated equipment or circuitries that are configured to acquire such signal waveforms for the purpose of monitoring health and/or diagnosing disease or abnormal conditions. The photoplethysmographic signal(s) typically include a red photoplethysmographic signal (e.g., an electromagnetic signal in the visible light spectrum most dominantly having a wavelength of approximately 625 to 740 nanometers) and an infrared photoplethysmographic signal (e.g., an electromagnetic signal extending from the nominal red edge of the visible spectrum up to about 1 mm), though other spectra such as near-infrared, blue and green may be used in different combinations, depending on the type and/or mode of PPG being employed.

The term “ballistocardiographic signal,” as used herein, refers to a signal or group of signals that generally reflect the flow of blood through the entire body that may be observed through vibration, acoustic, movement, or orientation. In some embodiments, ballistocardiographic signals are acquired by wearable devices, such as vibration, acoustic, movement, or orientation-based seismocardiogram (SCG) sensors, which can measure the body's vibrations or orientation as recorded by sensors mounted close to the heart. Seismocardiogram sensors are generally used to acquire “seismocardiogram,” which is used interchangeably with the term “ballistocardiogram” herein. In other embodiments, ballistocardiographic signals may be acquired by external equipment, e.g., bed or surface-based equipment that measures phenomena such as a change in body weight as blood moves back and forth in the longitudinal direction between the head and feet. In such embodiments, the volume of blood in each location may change dynamically and be reflected in the weight measured at each location on the bed as well as the rate of change of that weight.

In addition, the methods and systems described in the various embodiments herein are not so limited and may be utilized in any context of another physiological system or systems, organs, tissue, cells, etc., of a living body. By way of example only, two biophysical signal types that may be useful in the cardiovascular context include cardiac/biopotential signals that may be acquired via conventional electrocardiogram (ECG/EKG) equipment, bipolar wide-band biopotential (cardiac) signals that may be acquired from other equipment such as those described herein, and signals that may be acquired by various plethysmographic techniques, such as, e.g., photoplethysmography. In another example, the two biophysical signal types can be further augmented by ballistocardiographic techniques.

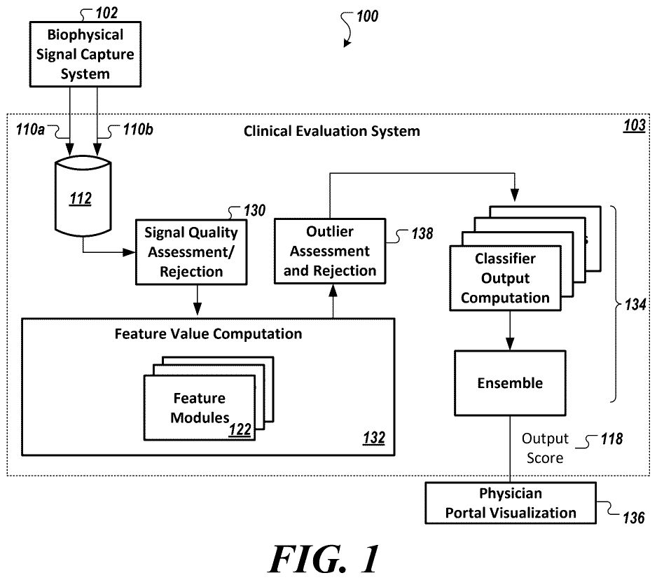

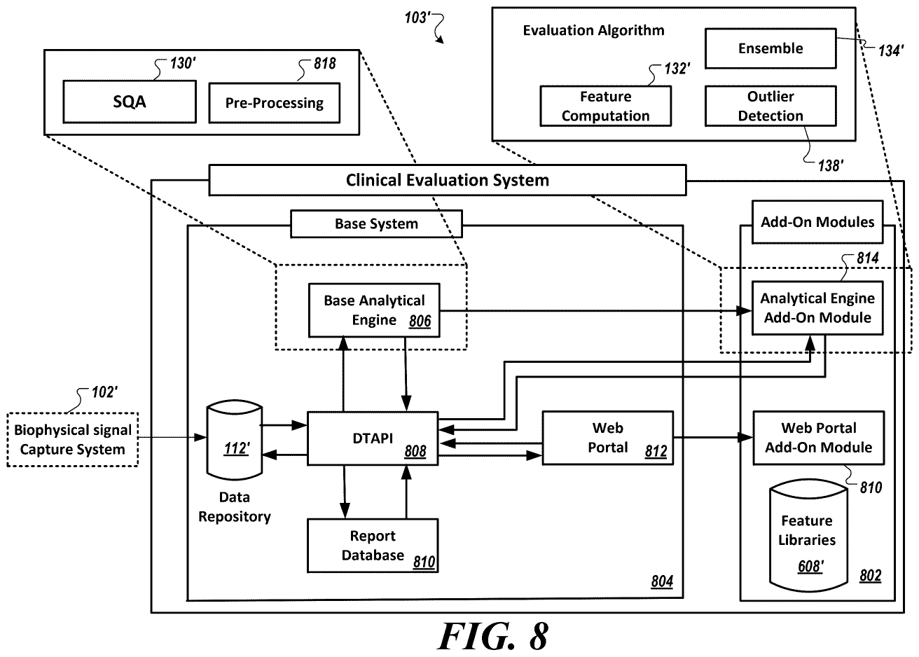

FIG. 1 shows a schematic diagram of the operation and workflow of an analytical engine or analyzer (e.g., 806, 814—see FIG. 8) of a clinical evaluation system 103 that can non-invasively estimate or determine a likelihood of the presence, non-presence, severity, and/or localization of a disease state, medical condition, or an indication of either in a patient. The analytical engines or analyzers include the operation and workflow for signal quality assessment and rejection (130), feature value computation (132), classifier output and ensemble computation and (134), physician portal visualization (136), and outlier assessment and rejection (138).

The analytical engine or analyzer is configured to non-invasively compute features or parameters to generate, via a classifier (e.g., machine-learned classifier), one or more metrics associated with the physiological state of a patient in accordance with an illustrative embodiment, e.g., to assist a healthcare provider in the diagnosis and/or treatment of cardiac- and cardiopulmonary-related pathologies and medical conditions, or an indicator of one. Examples include significant coronary artery disease (CAD), one or more forms of heart failure such as, e.g., heart failure with preserved ejection fraction (HFpEF), congestive heart failure, various forms of arrhythmia, valve failure, various forms of pulmonary hypertension, among various other disease and conditions disclosed herein.

In addition, there exist possible indicators of a disease or condition, such as an elevated or abnormal left ventricular end-diastolic pressure (PCWP) value as it relates to some forms of heart failure, abnormal left ventricular ejection fraction (LVEF) values as they relate to some forms of heart failure or an elevated mean pulmonary arterial pressure (mPAP) value as it relates to pulmonary hypertension and/or pulmonary arterial hypertension. Indicators of the likelihood that such indicators are abnormal/elevated or normal, such as those provided by the example analysis and classifiers described herein, can help a healthcare provider assess or diagnose that the patient has or does not have a given disease or condition. In addition to these metrics associated with a disease state of condition, other measurements and factors may be employed by a healthcare professional in making a diagnosis, such as the results of a physical examination and/or other tests, the patient's medical history, current medications, etc. The estimation of the presence or non-presence of a disease state or medical condition can include the indication (or a metric of estimation that is used in the diagnosis) for such disease.

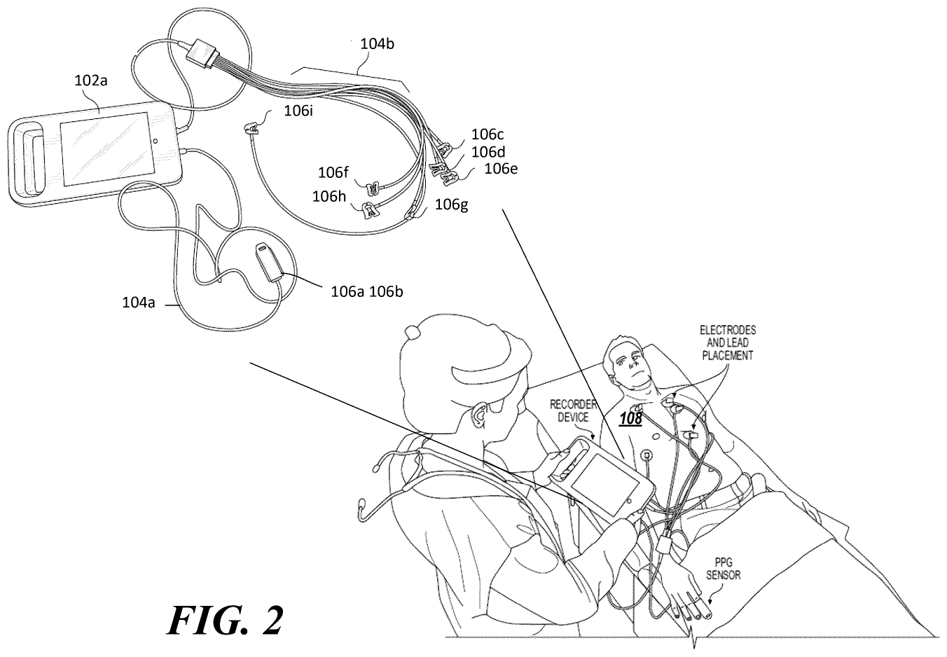

In FIG. 1, Clinical evaluation system 103 operates with a non-invasive biophysical signal recorder or capture system 102. The clinical evaluation system 103 may be implemented in cloud/remote infrastructure or in a local system. Biophysical signal capture system 102 (also referred to as a biophysical signal recorder system) is configured to, e.g., acquire, process, and store (and in certain implementations, transmit) synchronously acquired patient's electrical and hemodynamic signals as one or more types of biophysical signals 104 (not shown-see FIG. 2). In the example of FIG. 1, Biophysical signal capture system 102 is configured to synchronously capture two types of biophysical signals shown as first biophysical signals (e.g., synchronously acquired to other first biophysical signals) and second biophysical signals (e.g., synchronously acquired to the other biophysical signals) acquired from measurement probes 106 (e.g., not shown-see probes 106a and 106b, e.g., comprising hemodynamic sensors for hemodynamic signals 104a, and probes 106c-106h comprising leads for electrical/cardiac signals 104b in FIG. 2). The probes 106a-h are placed on, e.g., by being adhered to or placed next to, a surface tissue of a patient 108 (shown at patient locations 108a and 108b). The patient is preferably a human patient, but it can be any mammalian patient. The acquired raw biophysical signals (e.g., 106a and 106b) together form a biophysical-signal data set 110 (shown in FIG. 1 as a first biophysical-signal data set 110a and a second biophysical-signal data set 110b, respectively) that may be stored, e.g., as a single file, preferably, that is identifiable by a recording/signal captured number and/or by a patient's name and medical record number.

In the FIG. 1 embodiment, the first biophysical-signal data set 110a comprises a set of raw photoplethysmographic, or hemodynamic, signal(s) associated with measured changes in light absorption of oxygenated and/or deoxygenated hemoglobin from the patient at location 108a, and the second biophysical-signal data set 110b comprises a set of raw cardiac or biopotential signal(s) associated with electrical signals of the heart. Though in FIG. 1, raw photoplethysmographic or hemodynamic signal(s) are shown to be acquired at a patient's finger, the signals may be alternatively acquired at the patient's toe, wrist, forehead, earlobe, neck, etc. Similarly, although the cardiac or biopotential signal(s) are shown to be acquired via three sets of orthogonal leads, other lead configurations may be used (e.g., 11 lead configuration, 12 lead configuration, etc.).

The biophysical signal capture system 102 preferably acquires biophysical signals via non-invasive means or component(s). In alternative embodiments, invasive or minimally-invasively means or component(s) may be used to supplement or as substitutes for the non-invasive means (e.g., implanted pressure sensors, chemical sensors, accelerometers, and the like). In still further alternative embodiments, non-invasive and non-contact probes or sensors capable of collecting biophysical signals may be used to supplement or as substitutes for the non-invasive and/or invasive/minimally invasive means, in any combination (e.g., passive thermometers, scanners, cameras, x-ray, magnetic, or other means of non-contact or contact energy data collection system as discussed herein). Subsequent to signal acquisitions and recording, biophysical signal capture system 102 then provides, e.g., sending over a wireless or wired communication system and/or a network, the acquired biophysical-signal data sets 110a, 110b (or a data set derived or processed therefrom, e.g., filtered or pre-processed data) to a data repository 112 (e.g., a cloud-based storage area network) of the clinical evaluation system 103. In some embodiments, the acquired biophysical-signal data sets 110a, 110b are sent directly to the clinical evaluation system 103 for analysis or are uploaded to a data repository 112 through a secure clinician's portal. The data sets may be separately sent in multiple files or in a single data file. In other embodiments, the acquired biophysical-signal data sets 110a, 110b are analyzed by a clinical evaluation system executing on a local network computing device that can provide the results to a cloud/remote server.

Biophysical signal capture system 102 is configured with circuitries and computing hardware, software, firmware, middleware, etc., in some embodiments, to acquire, store, transmit, and optionally process both the captured biophysical signals to generate the biophysical-signal data set 110. An example biophysical signal capture system 102 and the acquired biophysical-signal set data 110 are described in U.S. Pat. No. 10,542,898, entitled “Method and Apparatus for Wide-Band Phase Gradient Signal Acquisition,” or U.S. Patent Publication No. 2018/0249960, entitled “Method and Apparatus for Wide-Band Phase Gradient Signal Acquisition,” each of which is hereby incorporated by reference herein in its entirety.

In some embodiments, biophysical signal capture system 102 includes two or more signal acquisition components, including a first signal acquisition component (not shown) to acquire the first biophysical signals (e.g., photoplethysmographic signals) and includes a second signal acquisition component (not shown) to acquire the second biophysical signals (e.g., cardiac signals). In some embodiments, the electrical signals are acquired at a multi-kilohertz rate for a few minutes, e.g., between 1 kHz and 10 kHz. In other embodiments, the electrical signals are acquired between 10 kHz and 100 kHz. The hemodynamic signals may be acquired, e.g., between 100 Hz and 1 kHz.

Biophysical signal capture system 102 may include one or more other signal acquisition components (e.g., sensors such as mechano-acoustic, ballistographic, ballistocardiographic, etc.) for acquiring signals. In other embodiments of the signal capture system 102, a signal acquisition component comprises conventional electrocardiogram (ECG/EKG) equipment (e.g., Holter device, 12 lead ECG, etc.).

Signal quality assessment/rejection (130). Analytical engine or analyzer (808, 814—see FIG. 8) assesses (130) the quality of the acquired biophysical-signal data set while the analysis pipeline is executing. The results of the assessment (e.g., pass/fail) may be performed, e.g., in a cloud-connected or network clinical evaluation system or a local version of one, and are immediately returned to the signal capture system's user interface for reading by the user. Acquired signal data that meet the signal quality requirements are deemed acceptable (i.e., “pass”) and may be further processed and subjected to analysis for the presence of metrics associated with the pathology or medical condition (e.g., elevated PCWP, CAD, PH/PAH, abnormal LVEF, HFpEF) by the analytical engine or analyzer. Acquired signals deemed unacceptable are rejected (e.g., “fail”), and a notification is immediately sent to the user to inform the user to immediately obtain additional signals from the patient 108 (see FIG. 2).

The analytical engine or analyzer can perform two sets of initial assessments for signal quality (130), one for the electrical signals and one for the hemodynamic signals. Operations (130) can confirm that the electrical signals (e.g., of the cardiac/biopotential signal data set 110b) are of sufficient length, lack high-frequency noise (e.g., above 170 Hz), and lack power line noise from the environment (e.g., 50 Hz or 60 Hz). An assessment of the hemodynamic signals can confirm (i) the percentage of outliers in the hemodynamic data set is below a pre-defined threshold and (ii) the percentage or maximum duration that the signals of the hemodynamic data set is railed, clipped, or saturated below a pre-defined threshold. Additional examples of signal quality assessments may be found in U.S. patent application Ser. No. 17/132,869, filed Dec. 23, 2020, entitled “Method and System for Signal Quality Assessment Using Heart Cycle Variability,” and U.S. Patent Publication no. 2020/0205739, entitled “Method and System for Automated Quantification of Signal Quality,” and PCT Publication no. WO2020/136570, entitled “Method and System for Automated Quantification of Signal Quality,” each of which is hereby incorporated by reference herein in its entirety.

Feature Value Computation (132). Analytical engine or analyzer (e.g., 806, 814—see FIG. 8) can compute (132) feature output values for a plurality of features modules 122 associated with a given clinical application. In the example of the PCWP algorithm, the analytical engine or analyzer can determine, in some embodiments, a total of 446 feature outputs belonging to multiple feature families implemented in Modules 122. The list of the features in the PCWP algorithm and description are provided in Table 1, later discussed herein.

Modules 122 may evaluate any number of features, including features that quantify waveform morphologies of biophysical signals or signals derived from the biophysical signals (e.g., morphologies of PPG signals and VPG/APG signals generated from the PPG signals and/or cardiac/biopotential signals). Modules 122 may evaluate features that quantify the spectral power of waveform regions in the biophysical signals. Modules 122 may evaluate features that quantify the spectral power of waveform regions of a cardiac/biopotential signal (e.g., ventricular depolarization, ventricular repolarization, and atrial depolarization regions) using wavelet analysis. Modules 122 may evaluate features that quantify propagative characteristics or visual topologic characteristics (e.g., velocity, trajectory, orbital frequency, planarity, vector length among registration points) in three-dimensional phase space of biophysical signals or regions in the biophysical signals. Modules 122 may evaluate features that quantify beat-to-beat variations (cycle variability) in a biophysical signal. Modules 122 may evaluate features that quantify synchronicity using Poincare plots among registration points in biophysical signals. Modules 122 may evaluate features that quantify dynamical characteristics of biophysical signals by way of dynamical system analysis. Modules 122 may evaluate features that quantify the power spectrum and frequency content of biophysical signals. Modules 122 may evaluate features that quantify the predictability of the signal. Modules 122 may evaluate features that assess biophysical signals in various phase space associated analyses (e.g., Phase Space Tomography), phase space models, phase space volumetric models.

Analytical engine or analyzer (e.g., 806, 814—see FIG. 8) may perform pre-processing and/or pre-conditioning operations (e.g., 818—see FIG. 8) to the signals. In some embodiments, the analytical engine or analyzer can process the acquired signals (e.g., cardiac signals) to remove transient time (e.g., the first 5 seconds, 10 seconds 20 seconds, 30 seconds, 31 seconds, 45 seconds, 1 minute, etc.) of the signal and omit or remove them from subsequent analysis, e.g., in case such portion of the signal was obtained while any initial motion of the patient at the start of the signal acquisition procedure may have occurred and/or while the electrode or sensor contact settles or initially shifts. The analytical engine or analyzer may remove the direct current (DC) offset of the acquired signal, e.g., by subtracting the acquired signal (e.g., cardiac signals) from its mean for each acquired channel. The analytical engine or analyzer may remove an assessed baseline wander, e.g., by subtracting the acquired signal (e.g., cardiac signals) from its low-frequency baseline. In some embodiments, the baseline is generated by a reconstruction operation that uses a stationary wavelet transform (e.g., Daubechies wavelet with a number of vanishing moments, e.g., 8 moments) that preserves its coefficients at the lowest frequency.

Outlier Assessment and Rejection Detection (138). Following the computation of the feature value outputs (in process 132) and prior to their application to the classifier models (in process 134, Analytical engine or analyzer (e.g., 806, 814—see FIG. 8) can perform outlier analysis (shown in process 138) of the feature value outputs. Outliers are signals (e.g., cardiac and/or photoplethysmographic signals) that are unlikely to be observed or expected to be observed in the trained dataset. It is desirable to remove outliers because, by the definition of an outlier as unlikely to be observed, the training or assessment system has very limited exposure to such signals, and therefore the possibility of prediction error is higher on an outlier signal as compared to a non-outlier signal.

The outlier detection module assesses for outliers that present themselves within sparse clusters at isolated regions that are out of distribution from the rest of the observations. Process 138 can reduce the risk that outlier signals are inappropriately applied to the classifier models and produce inaccurate estimates that would otherwise be viewed by the patient or healthcare provider.

Outlier analysis evaluation process 138 can execute an outlier detection module (ODM) to identify and exclude anomalous acquired biophysical signals to prevent the generation of a final score that has a higher likelihood of error due to outlier signals. Outlier analysis evaluation process 138 can employ an unsupervised learning algorithm (e.g., Isolation Forest or other decision tree-based methods) for anomaly detection. Outlier analysis evaluation process 138 can isolate samples through the selection of random features and randomly splitting points between the maximum and minimum values of that feature. In the example of an isolation forest, operation 138 can employ the natural tendency for outliers to separate from the rest of the observations with a few steps through the trees (shorter average pathway). The ‘forest’ algorithm, in some embodiments, is configured to generate a collection of decision trees and to make a final decision based on all the trees within the forest.

Two categories of anomalous signals may be evaluated, among others, by the outlier detection model (ODM), including arrhythmic data sets and inverted lead data sets. Arrhythmic data sets, in some embodiments, are labeled based on one or more indicators, including (i) sudden changes in heart rates or (ii) missing or extra waveforms. Sudden changes in heart rate may be defined as a burst of fast cycles (paroxysmal tachycardia) or a sudden change from one reasonable heart rate to another. An example includes a determined change from 72 bpm to 48 bpm, which is a 2:1 change in the AV node block signal. Missing or extra waveforms may be defined as a presence and/or absence of any of the depolarization and/or repolarization waves, e.g., ventricular or atrial depolarization waveforms (also referred to as a QRS wave or P-waves). Inverted leads (e.g., as caused by misplacement of the electrodes on the torso) can be a clinical study protocol deviation in the collection of the data set that can generate anomalous signals to which hyperparameters of ODM can be tuned to encourage its detection.

In an example, the ODM operator can be trained using an ensemble of isolation forests (e.g., 50 models) on a data set consisting of 1000+ signals, while hyperparameter tuning was performed on a labeled test set consisting of signals with two categories of arrhythmia and inverted leads. To enhance the ODM robustness to the noise in the dataset, models can be trained each on the 80% of randomly selected samples from the training data and the rest used for testing. The ODM prediction may be based on ensemble voting (the arithmetic mean of the outlier scores of each model). During the hyperparameters tuning, the ODM operator is evaluated to identify all the labeled anomalous signals in a test set with an acceptable outlier detection rate (ODR) generalization. The results can be confirmed using a hold-out validation set. Additional examples and descriptions of other isolation forest methods are described in Liu, Fei Tony, Ting, Kai Ming and Zhou, Zhi-Hua. “Isolation forest.” Data Mining, 2008. ICDM′08. Eighth IEEE International Conference (2008), which is incorporated by reference herein in its entirety.

Classifier Output Computation (1334). Analytical engine or analyzer (e.g., 806, 814—see FIG. 1) then uses the calculated feature outputs in classifier models (e.g., machine-learned classifier models) to generate a set of model scores. Classifier models may include transfer functions, look-up tables, models, or operators developed based on algorithms such as but not limited to decision trees, random forests, neural networks, linear models, Gaussian processes, nearest neighbor, SVMs, Naïve Bayes, etc. The analytical engine or analyzer can join the set of model scores in an ensemble of the constituent models, which, in some embodiments, averages the output of the classifier models as shown in Equation 1.

$$Ensemble estimation = \frac{{Model}_{1} + {Model}_{2} + … + {Model}_{n}}{n}$$

### (Equation 1)

In some embodiments, classifier models may include models that are developed based on ML techniques described in U.S. Patent Publication No. 20190026430, entitled “Discovering Novel Features to Use in Machine Learning Techniques, such as Machine Learning Techniques for Diagnosing Medical Conditions”; or U.S. Patent Publication No. 20190026431, entitled “Discovering Genomes to Use in Machine Learning Techniques,” each of which is hereby incorporated by reference herein in its entirety.

In the example of the PCWP algorithm, thirteen (13) machine-learned classifier models are each calculated using the calculated feature outputs. The 13 classifier models include four ElasticNet machine-learned classifier models [9], four RandomForestClassifier machine-learned classifier models [10], and five extreme gradient boosting (XGB) classifier models [11]. In some embodiments, the patient's metadata information, such as age, gender, and BMI value, may be used. The output of the ensemble estimation may be a continuous score. The score may be shifted to a threshold value of zero by subtracting the threshold value for presentation within the web portal. The threshold value may be selected as a trade-off between sensitivity and specificity. The threshold may be defined within the algorithm and used as the determination point for test positive (e.g., “Likely Elevated PCWP”) and test negative (e.g., “Not Likely Elevated PCWP”) condition.

In some embodiments, the analytical engine or analyzer can fuse the set of model scores with a body mass index-based adjustment or an adjustment based on age or gender. For example, the analytical engine or analyzer can average the model estimation with a sigmoid function of the patient BMI having the form

$$sigmoid ( x ) = \frac{1}{1 + {e}^{- x}} .$$

Physician Portal Visualization (136). The patient's report may include a visualization 136 of the acquired patient data and signals and the results (e.g., shown as an output score 118 in an example) of the disease analyses. The analyses may be presented in multiple sections within the report, including a section that shows the score, a section that shows the score with respect to a positive indication population group and a negative indication group, and a section that shows the specificity and sensitivity performance of the score. A healthcare provider, e.g., a physician, can review the report and interpret it to provide a diagnosis of the disease or to generate a treatment plan. The healthcare portal may list a report for a patient if a given patient's acquired signal data set meets the signal quality standard. The report may indicate a disease-specific result, e.g., (i) estimation of the presence or non-presence of elevated indication of a disease or condition (e.g., elevated PCWP or mPAP or other indications discussed herein) or (ii) presence, non-presence, severity, or localization of a disease or medical condition (e.g., CAD, PH, PAH, or other disease or conditions as described herein).

The output report may be via a wearable device, a handheld device, or a medical diagnostic equipment (e.g., pulse oximeter system, wearable health monitoring systems). In some embodiments, the output may be via a point of care monitoring, such as a mobile cart or cart trolley. In some embodiments, the outputs may be used in resuscitation systems, cardiac or pulmonary stress test equipment, pacemakers, etc., in which frequency spectral information is desired.

### Example Biophysical Signal Acquisition.

FIG. 2 shows a biophysical signal capture system 102 (shown as 102a) and its use in non-invasively collecting biophysical signals of a patient in a clinical setting in accordance with an illustrative embodiment. In FIG. 2, the biophysical signal capture system 102a is configured to capture two types of biophysical signals from patient 108 while the patient is at rest. Biophysical signal capture system 102a synchronously acquires the patient's (i) electrical signals (e.g., cardiac signals corresponding to the second biophysical-signal data set 110b) from the torso using orthogonally placed sensors (106c-106h; 106i is a common-mode reference lead) and (ii) hemodynamic signals (e.g., PPG signals corresponding to the first biophysical-signal data set 110a) from the finger using a photoplethysmographic sensor (e.g., collecting signals 106a, 106b).

As shown in FIG. 2, the electrical and hemodynamic signals (e.g., 104a, 104b) are passively collected via commercially available sensors applied to the patient's skin. The signals may be acquired beneficially without patient exposure to ionizing radiation or radiological contrast agents and without patient exercise or the use of pharmacologic stressors. The biophysical signal capture system 102a can be used in any setting conducive for a healthcare professional, such as a technician or nurse, to acquire the requisite data and where a cellular signal or Wi-Fi connection can be established.

The electrical signals (e.g., corresponding to the second biophysical signal data set 110b) are collected using three orthogonally paired surface electrodes arranged across the patient's chest and back along with a reference lead. The electrical signals are acquired, in some embodiments, using a low-pass anti-aliasing filter (e.g., ~2 kHz) at a multi-kilohertz rate (e.g., 8 thousand samples per second for each of the six channels) for a few minutes (e.g., 215 seconds). In alternative embodiments, the biophysical signals may be continuously/intermittently acquired for monitoring, and portions of the acquired signals are used for analysis. The hemodynamic signals (e.g., corresponding to the first biophysical signal data set 110a) are collected using a photoplethysmographic sensor placed on a finger. The photo-absorption of red light (e.g., any wavelengths between 600-750 nm) and infrared light (e.g., any wavelengths between 850-950 nm) are recorded, in some embodiments, at a rate of 500 samples per second over the same period. The biophysical signal capture system 102a may include a common mode drive that reduces common-mode environmental noise in the signal. The photoplethysmographic and cardiac signals were simultaneously acquired for each patient. Jitter (inter-modality jitter) in the data may be less than about 10 microseconds (ms). Jitter among the cardiac signal channels may be less than 10 microseconds, e.g., around ten femtoseconds (fs).

A signal data package containing the patient metadata and signal data may be compiled at the completion of the signal acquisition procedure. This data package may be encrypted before the biophysical signal capture system 102a transfers to the data repository 112. In some embodiments, the data package is transferred to the clinical evaluation system (e.g., 103). The transfer is initiated, in some embodiments, following the completion of the signal acquisition procedure without any user intervention. The data repository 112 is hosted, in some embodiments, on a cloud storage service that can provide secure, redundant, cloud-based storage for the patient's data packages, e.g., Amazon Simple Storage Service (i.e., “Amazon S3”). The biophysical signal capture system 102a also provides an interface for the practitioner to receive notification of an improper signal acquisition to alert the practitioner to immediately acquire additional data from the patient.

### Example Method of Operation

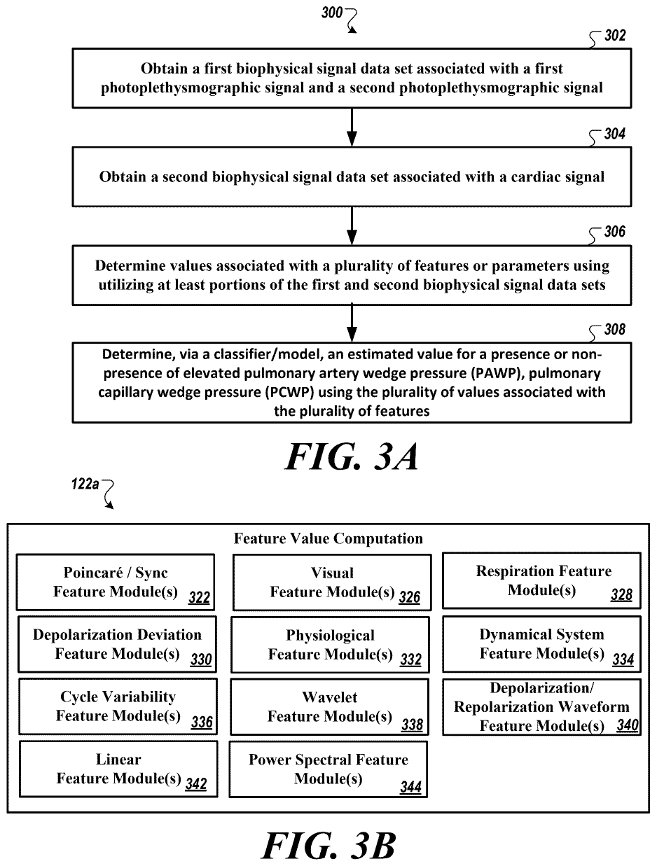

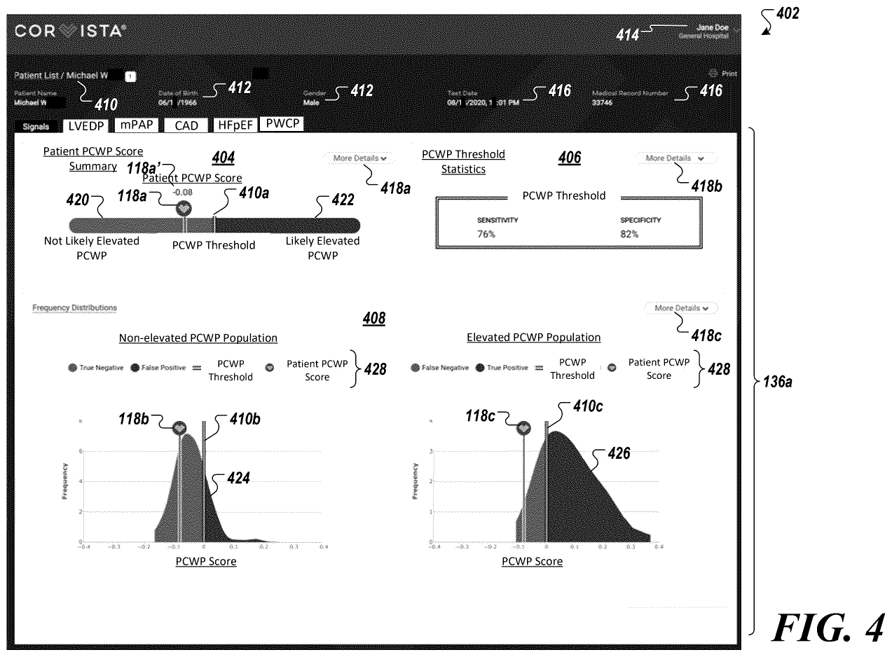

FIG. 3 shows an example method 300 for non-invasively determining an estimation for the presence of elevated PCWP. Method 300 may additionally determine an estimation for the non-presence of elevated PCWP to assess in combination with the positive determination. Method 300 includes the step of obtaining (302) a first biophysical signal data set associated with a first photoplethysmographic signal and a second photoplethysmographic signal, e.g., as described in relation to FIGS. 1 and 2 and other examples described herein. In some embodiments, the acquired biophysical signal data set is transmitted for remote storage and analysis. In other embodiments, the acquired biophysical signal data set is stored and analyzed locally. The first biophysical data set may have been acquired over multiple cardiac cycles of the patient.

Method 300 further includes the step of obtaining (304) a second biophysical signal data set associated with a cardiac signal, wherein the second biophysical data set has been simultaneously acquired with the first biophysical signal data set over the multiple cardiac cycles.

Method 300 further includes the step of determining (306), utilizing at least a portion of the first and second biophysical signal data sets, a plurality of values associated with a plurality of features. Method 300 further includes the step of determining (308) an estimate in the form of a numerical score or value relating to the likelihood that the patient has an elevated PCWP using the plurality of values associated with the plurality of features assessing synchronicity between the cardiac signal and the photoplethysmographic signal and features associated with respective cardiac signal and the photoplethysmographic signal. The estimated value (e.g., as an elevated PCWP score) for the likelihood the patient has an elevated PCWP may be outputted for use in a diagnosis and/or treatment of a disease or condition such as, e.g., one or more forms of heart failure.

FIG. 3B shows an example of feature module(s) 122 (shown as 122a) which can be used in the PCWP assessment operation and workflow. Modules 122 provide a pool of features that can be assessed to provide the best estimate for the assessment of elevated PCWP. The number of features and feature classes may be adjusted while still being able to provide an estimation.

TABLE 1

| Name | Description |
|---|---|
| Poincare/Synchronicity | Features calculated using the infrared component of the PPG |
| (e.g., module 322) | signal, where the time difference between the peaks of the |
| Features = 6 (Table 2A) | waveform are input to a Poincare analysis to quantify the |
| | signal dynamics. Features may be calculated using both |
| | components of the PPG signal (1st modality) as well as all |
| | three channels of the biopotential signal (2nd modality) to |
| | characterize the synchronicity between the two modalities |
| | using Poincare maps. |
| Visual | Features calculated using the three channels of the OVG |
| (e.g., module 326) | signal in three-dimensional phase space. In phase space, |
| Features = 66 (34 + 32 + 25) | atrial depolarization, ventricular depolarization, and |
| (Tables 2B, 2C) | ventricular repolarization form three distinct loops. Features |
| | are comprised of various geometric measures for each loop |
| | as well as the interaction of a given loop with respect to the |
| | others. |
| | For PPG signal, features are calculated using the red and |
| | infrared components of each of the PPG, VPG, and APG, |
| | which are combined into three-dimensional phase spaces, |
| | and in some cases, projected into two-dimensional phase |
| | spaces. Features also calculated using the three channels of |
| | the biopotential signal in three-dimensional phase space. In |
| | phase space, atrial depolarization, ventricular depolarization, |
| | and ventricular repolarization form three distinct loops. |
| | Features are comprised of various geometric and other |
| | measures for the data in these phase spaces. |
| | An extension of the OVG Visual set intended to |
| | specifically capture aspects of left ventricular hypertrophy, |
| | left atrial enlargement and hypertrophic cardiomyopathy. |
| Respiration | Features to approximate the respiration waveform, and |
| (e.g., module 328) | evaluate characteristics of that approximation, using all the |
| Respiration = 3 (Table 2D) | PPG and biopotential signals. Modulation signals are |
| | produced on each input signal, and merged, then features are |
| | extracted including 1) heart rate variability from modulation |
| | signals, 2) respiration rate information from respiration rate |
| | signals, 3) discrepancy features representing the distance |
| | between respiration and modulation signals, 5) square |
| | coherence representing the correlation between modulation |
| | and respiration rate signals. |
| Depolarization Deviation | Features that quantify deviations of the VD wave trajectory |
| (e.g., module 330) | from the trajectory of the three-dimensional modeled VD |
| Feature = 1 (Table 2E) | wave. The model wave is a representation of the VD in a |
| | lower-dimensional space embedding the most prominent |
| | frequency content of the original signal below 40 Hz. These |
| | features are specifically engineered to evaluate the high- |
| | frequency & low-amplitude patterns in the VD trajectory. |
| Physiological | Features quantifying specific physiological aspects of the |
| (e.g., module 332) | biopotential signal. |
| Feature = 6 (Table 2F) | |
| Dynamical Systems | Features to quantify dynamical characteristics of the three |
| (e.g., module 334) | biopotential channels and PPG signals, including Lyapunov |
| Features = 4 (Table 2G) | exponent, correlation dimension, entropy, mutual |
| | information, correlation, and nonlinear filtering. |
| Cycle Variability | Features to quantify the beat-to-beat variations in |
| (e.g., module 336) | biopotential by comparing each beat to the template beat. |
| Feature = 2 (Table 2H) | The template beat is the most prominent waveform across |
| | the entire signal, obtained by applying the median filter to |
| | the stacked beat-to-beat segmented signal via VD peak |
| | matching. |
| Wavelet | Features extracted using a wavelet analysis of the signal, |
| (e.g., module 338) | specifically the ventricular depolarization, ventricular |
| Features = 57 (15 + 42) | repolarization, and atrial depolarization. |
| (Tables 2I, 2J) | Features extracted using a wavelet analysis of the |
| | PPG signal, specifically the systolic and diastolic waveform. |
| Depolarization/ | Depolarization Features to quantify the propagative |
| Repolarization Waveform | characteristics of the ventricular depolarization (VD) wave |
| (e.g., module 340) | in three-dimensional space. The main wave characteristics |
| Features = 35 (22 + 13 + 10) | are categorized in four groups based on the 1. velocity, 2. |
| (Tables 2K, 2L) | trajectory, 3. orbital frequency (3D rotation rate), and 4. |
| | planarity of the wave. In addition to VD wave, the wave |
| | propagation features are also applied to the bandpass |
| | filtered VD waves at various frequency sub-bands. |
| | Repolarization: Similar to Depolarization Wave |
| | Propagation, these features are to quantify the propagative |
| | characteristics of ventricular repolarization (VR) wave in |
| | three-dimensional space. The main wave characteristics are |
| | categorized in four groups based on the 1. velocity, 2. |
| | trajectory, 3. orbital frequency (3D rotation rate), and 4. |
| | planarity of the wave. In addition to VR wave, the wave |
| | propagation features are also applied to the bandpass |
| | filtered VR waves at various frequency sub-bands. |
| Linear | Features that quantify both conventional cardiac |
| (e.g., module 342) | measurements and variations, using the three biopotential |
| Features = 13 (8 + 5) (Tables | channels, such as waveform amplitudes, durations, heart |
| 2M, 2N) | rate, and morphologies. |
| Linear Ventricular | Features to quantify properties of the PPG, as well as a |
| Hypertrophy (LVH) | velocity photoplethysmogram (1st time derivative of the |
| (e.g., module 346) | PPG; “VPG”) and an acceleration photoplethysmogram (2nd |
| Features = 5 | time derivative of the PPG; “APG”), repeated for the red and |
| | infrared components of the PPG. Features are calculated |
| | using peak amplitudes, peak-to-peak distances, angles |
| | between points, and various ratios. |
| | An extension of the OVG Linear set intended to |
| | specifically capture aspects of left ventricular hypertrophy. |
| Power Spectral | Features to quantify the power spectrum and frequency |
| (e.g. module 344) | content of the PPG or cardiac signals. These features are |
| Feature = 1 (Table 2O) | designed to study the PPG in the frequency domain by |
| | obtaining the power spectrum and coherence (cross-spectral |
| | analysis). |

Poincare/Synchronicity. The first feature set, per Table 2A, is directed to calculations using the infrared component of the PPG signal, where the time difference between the peaks of the waveform are input to a Poincare analysis to quantify the signal dynamics.

Example descriptions of the features may be found in U.S. Patent Application no. US2020/0397322-A1, entitled “Method and System to Assess Disease Using Dynamical Analysis of Biophysical Signals,” which is incorporated by reference herein in its entirety.

In Table 2A, a feature dYMode2 refers to a mode of distribution of the PSR/ECG “Y” channel data triggered at second crossover landmarks of photoplethysmographic signals (e.g., in Poincaré map analysis PM #1). A feature dXModel refers to a mode of distribution of the PSR/ECG “X” channel data triggered at the second crossover landmarks of photoplethysmographic signals. A feature dMeanL refers to a mean value of histogram of Poincaré map analysis “PM2” of infrared photoplethysmographic signal.

TABLE 2A

| | |
|---|---|
| sync_duo_dYMode2_upper_lower_orth2 | sync_duo_dMeanL_lower |
| sync_duo_dXMode1_upper_lower_orth1 | sync_duo_dSkewL_lower |
| sync_duo_dZRelStdMAD2_upper_{—} | pc_ppg_axisRatio_{—} |
| lower_orth3 | upper_lower |

Visual Features. The second feature set, per Table 2B and Table 2C, is directed to calculations using the three channels of the OVG signal in three-dimensional phase space. In phase space, atrial depolarization, ventricular depolarization, and ventricular repolarization form three distinct loops. Features are comprised of various geometric measures for each loop as well as the interaction of a given loop with respect to the others.

For PPG signals, the features are calculated using the red and infrared components of each of the PPG, VPG, and APG, which are combined into three-dimensional phase spaces, and in some cases, projected into two-dimensional phase spaces. Features also calculated using the three channels of the biopotential signal in three-dimensional phase space. In phase space, atrial depolarization, ventricular depolarization, and ventricular repolarization form three distinct loops.

Example descriptions of the features may be found in U.S. Patent Application no. US2023/0071085, entitled “Methods and Systems for Engineering Visual Features From Biophysical Signals for Use in Characterizing Physiological Systems,” which is incorporated by reference herein in its entirety. A feature MVRV_cosAzimuth refers to an angle of a 2D projection of 3D vector in 3D phase space to a pre-defined axis of each vector of the maximal ventricular repolarization vector (MVRV). A feature MVRV_cos 2Dangle refers to the angle of a projection vector makes with the horizontal axis for a given orthogonal plane, e.g., in degrees. A feature 2dArea_Quad2 refers to an area of the quadrant (4), or overall, in each of the three orthogonal projections of waveform regions of a cardiac signal or a PPG signal. A feature TVDV_MVRV_2dAngle refers to an area of the three orthogonal projections of waveform regions between a terminal ventricular depolarization vector and the maximal ventricular repolarization vector. A feature 2dPerimeter refers to a mode of the sum of Euclidean distances of consecutive paired points for each cycle in quadrants or the overall orthogonal projection of waveform regions of a cardiac signal or a PPG signal.

TABLE 2B

| | |
|---|---|
| visual_ovg_MVRV_cosAzimuth_orth123 | visual_ovg_MVRV_2DAngle_orth13 |
| visual_ovg_MVRV_cos2DAngle_orth13 | visual_ovg_VD_2dArea_Quad2_orth13 |
| visual_ovg_VR_2dPerimeter_Quad3_orth12 | visual_ovg_MVRV_2DAngle_orth12 |
| visual_ovg_TVDV_MVRV_2dAngle_orth12 | visual_ovg_VD_2dArea_Quad3_orth23 |
| visual_ovg_MVRV_2DQuadNum_orth12 | visual_ovg_VR_2dArea_Quad2_orth12 |
| visual_ovg_MVRV_2DQuadNum_orth13 | visual_ovg_VR_2dArea_Quad2_orth23 |
| visual_ovg_VR_2dPerimeter_Quad3_orth23 | visual_ovg_MVRV_2DAngle_orth13 |
| visual_ovg_MVRV_OctantNum_orth123 | visual_ovg_VD_Eccentricity_orth13 |
| visual_ovg_VR_2dPerimeter_Quad2_orth12 | visual_ovg_AD_MaxVelocity_orth123 |
| visual_ovg_IVDV_MVRV_2dAngle_orth13 | visual_ovg_VR_Max2dPerimeterQuadNum_orth13 |
| visual_ovg_TVDV_MVRV_sin2dAngle_orth12 | visual_ovg_TVDV_MVRV_2dAngle_orth13 |
| visual_ovg_MVDV_MVRV_cos3dAngle_orth123 | visual_ovg_MVDV_MVRV_2dAngle_orth13 |
| visual_ovg_MVDV_MVRV_cos2dAngle_orth12 | visual_ovg_TVDV_MVRV_2dAngle_orth13 |
| visual_ovg_TVDV_MVRV_sin2dAngle_orth13 | visual_ovg_VR_Max2dAreaQuadNum_orth13 |
| visual_ovg_VD_Max2dPerimeterQuadNum_orth23 | visual_ovg_VR_Max2dPerimeterQuadNum_orth13 |
| visual_ovg_IVDV_MVRV_sin2dAngle_orth13 | visual_ovg_AD_2dPerimeter_Quad3_orth12 |
| visual_ovg_MVDV_MVRV_2dAngle_orth12 | visual_ovg_VR_2dPerimeter_Quad3_orth13 |

In Table 2C, a feature ppg_peakVPG_2DAngle refers to a 2D angel between a PPG signal and a APG signal. A feature 2DAmplitude refers to a magnitude of a projection of a given vector in a given orthogonal plane (e.g., between PPG, VPG, and APG signals).

TABLE 2C

| | |
|---|---|
| visual_ppg_peakVPG_2DAngle_PPGAPG_lower | visual_ppg_baseVPG_2DAmplitude_PPGVPG_upper |
| visual_ppg_peakVPG_sin2DAngle_PPGAPG_lower | visual_ppg_baseAPG_2DAmplitude_PPGAPG_upper |
| visual_ppg_peakVPG_cos2DAngle_PPGAPG_lower | visual_ppg_peakVPG_2DAmplitude_PPGAPG_lower |
| visual_ppg_baseVPG_2DAmplitude_PPGVPG_lower | visual_ppg_peakAPG_sin2DAngle_VPGAPG_lower |
| visual_ppg_peakVPG_2DAngle_PPGVPG_upper | visual_ppg_minAPG_cos2DAngle_PPGVPG_upper |
| visual_ppg_peakVPG_sin2DAngle_PPGAPG_upper | visual_ppg_baseVPG_2DAmplitude_PPGAPG_upper |
| visual_ppg_peakVPG_2DAmplitude_PPGAPG_upper | visual_ppg_peakVPG_sin2DAngle_PPGVPG_upper |
| visual_ppg_diasPPG_2DAngle_PPGAPG_lower | visual_ppg_peakVPG_cos2DAngle_PPGVPG_upper |
| visual_ppg_diasPPG_cos2DAngle_PPGAPG_lower | visual_ppg_minAPG_sin2DAngle_PPGVPG_upper |
| visual_ppg_peakAPG_cos2DAngle_VPGAPG_lower | visual_ppg_minAPG_sin2DAngle_PPGVPG_lower |
| visual_ppg_peakAPG_cos2DAngle_VPGAPG_upper | visual_ppg_minAPG_2DAngle_PPGVPG_lower |
| visual_ppg_diasPPG_cos2DAngle_PPGAPG_upper | visual_ppg_minVPG_sin2DAngle_PPGAPG_lower |
| visual_ppg_3dMaxCurvature_lower | visual_ppg_minAPG_2DAngle_PPGVPG_upper |
| visual_ppg_baseAPG_sin2DAngle_VPGAPG_upper | visual_ppg_peakAPG_3DAmplitude_lower |
| visual_ppg_baseVPG_2DAmplitude_PPGAPG_lower | visual_ppg_diasPPG_cosElevation_upper |
| visual_ppg_minAPG_cos2DAngle_PPGVPG_lower | visual_ppg_diasPPG_cosElevation_lower |
| visual_ppg_baseAPG_3DAmplitude_lower | visual_ppg_peakAPG_2DAngle_VPGAPG_lower |
| lvedpvisual_ovg_detlaInitialTerminalQRSDuration_orth123 | lvedpvisual_ovg_meanS_sin_AngleFP_orth12 |
| lvedpvisual_ovg_meanQRS_G_cos_AngleHPorth13 | lvedpvisual_ovg_meanQRS_T_AngleSP_orth23 |
| lvedpvisual_ovg_maxQRS_T_cos_AngleFP_orth12 | lvedpvisual_ovg_meanQRS_T_cos_AngleSP_orth23 |
| lvedpvisual_ovg_maxQRS_T_cos_AngleHP_orth13 | lvedpvisual_ovg_maxTcos_AngleSP_orth23 |
| lvedpvisual_ovg_meanQRS_sin_AngleFP_orth12 | lvedpvisual_ovg_meanQRS_T_cos_AngleFP_orth12 |
| lvedpvisual_ovg_meanQRS_T_sin_AngleSP_orth23 | lvedpvisual_ovg_maxQRS_sin_AngleFP_orth12 |
| lvedpvisual_ovg_maxTcos_AngleHP_orth13 | lvedpvisual_ovg_meanQRS_sin_AngleHP_orth13 |
| lvedpvisual_ovg_meanT_cos_AngleHP_orth13 | lvedpvisual_ovg_meanQRS_T_Angle3d_orth123 |
| lvedpvisual_ovg_meanT_AngleHP_orth13 | lvedpvisual_ovg_meanQRS_T_sin_Angle3d_orth123 |
| lvedpvisual_ovg_maxQRS_sin_AngleHP_orth13 | lvedpvisual_ovg_meanQRS_T_cos_AngleHP_orth13 |
| lvedpvisual_ovg_meanQRS_AngleHP_orth13 | lvedpvisual_ovg_maxTAngleFP_orth12 |
| lvedpvisual_ovg_meanT_azimuth_orth123 | lvedpvisual_ovg_maxTAngleHP_orth13 |
| lvedpvisual_ovg_maxTazimuth_orth123 | |

Respiration Features. The third feature set, per Table 2D, is directed to features to approximate the respiration waveform, and evaluate characteristics of that approximation, using all the PPG and biopotential signals. Modulation signals are produced on each input signal, and merged, then features are extracted including 1) heart rate variability from modulation signals, 2) respiration rate information from respiration rate signals, 3) discrepancy features representing the distance between respiration and modulation signals, 5) square coherence representing the correlation between modulation and respiration rate signals.

Example descriptions of the features may be found in U.S. Patent Application no. US2023/0127355A1, entitled “Method and System for Engineering Respiration Rate-Related Features From Biophysical Signals for Use in Characterizing Physiological Systems,” which is incorporated by reference herein in its entirety. A feature dPkMMDStd refers to standard deviation of the distribution of assessed maximum mean discrepancy distance estimations between PDF of a PPG signal and a power density. A feature dPkCXYMean refers to mean of the distribution of assessed coherence estimations between a modulated PPG signal and an estimated respiration waveform where the PPG signal is modulated by peak modulation. A feature dRRMean refers to the mean of the distribution of respiration rate estimations.

TABLE 2D

| | |
|---|---|
| resp_duo_dPkMMDStd_upper_lower | resp_duo_dPkCXYMean_{—} |
| | upper_lower |
| resp_duo_dRRMean_upper_lower_{—} | |
| orth123 | |

Depolarization Deviation Features. The fourth feature set, per Table 2E, is directed to features that quantify deviations of the VD wave trajectory from the trajectory of the three-dimensional modeled VD wave. The model wave is a representation of the VD in a lower-dimensional space embedding the most prominent frequency content of the original signal below 40 Hz. These features are specifically engineered to evaluate the high-frequency & low-amplitude patterns in the VD trajectory.

Example descriptions of the features may be found in U.S. Patent Application no. US2023/0075634A1, entitled “Methods and Systems for Engineering Conduction Deviation Features From Biophysical Signals for Use in Characterizing Physiological Systems,” which is incorporated by reference herein in its entirety. A feature medianCondDelay refers to a median of the accumulative conduction distance.

TABLE 2E

dwpdev_ovg_medianCondDelay_3D_orth123

Physiological Features. The fifth feature set, per Table 2F, is directed to Features quantifying specific physiological aspects of the biopotential signal.

Example descriptions of the features may be found in U.S. Patent Application no. US2023/0076069A1, entitled “Methods and Systems for Engineering Cardiac Waveform Features From Biophysical Signals for Use in Characterizing Physiological Systems,” which is incorporated by reference herein in its entirety. A feature ampIntialPeak_I refers to an amplitude of the first peak (e.g., initial peak) for signal I. A feature morphology refers to a morphology shape. A feature dist_terminalSegment refers to a distribution of the second segment.

TABLE 2F

| | |
|---|---|
| phys_ovg_dower_ampIntialPeak_I | phys_ovg_dower_dist_terminalSegment_V1 |
| phys_ovg_femaleCluster3_oneHot_orth123 | phys_ovg_dist_terminalSegment_orth2 |
| phys_ovg_ampIntialPeak_orth1 | phys_ovg_morphology_orth2 |

Dynamical Systems Features. The sixth feature set, per Table 2G, is directed to features to quantify dynamical characteristics of the three biopotential channels and PPG signals, including Lyapunov exponent, correlation dimension, entropy, mutual information, correlation, and nonlinear filtering.

Example descriptions of the features may be found in U.S. Pat. No. 11,291,379, entitled “Method and System to Assess Disease Using Dynamical Analysis of Cardiac and Photoplethysmographic Signals,” which is incorporated by reference herein in its entirety.

TABLE 2G

| | |
|---|---|
| dyn_ppg_CsLEX_orth1 | dyn_ovg_LEY_orth2 |
| dyn_ppg_SpCsXCFUXMax_upper_{—} | dyn_ovg_ACFZZ2_orth3 |
| orth1 | |
| dyn_ppg_SpAMILmin_lower | dyn_ovg_ACFYZ1_orth23 |
| dyn_ppg_SpAMIUmin_upper | dyn_ovg_K2Y_orth2 |
| dyn_ppg_SpCsXCFUX1_upper_orth1 | |

Cycle Variability Features. The seventh feature set, per Table 2H, is directed to features to quantify the beat-to-beat variations in biopotential by comparing each beat to the template beat. The template beat is the most prominent waveform across the entire signal, obtained by applying the median filter to the stacked beat-to-beat segmented signal via VD peak matching.

Example descriptions of the features may be found in U.S. Pat. No. 12,324,683, titled “Method and System to Assess Disease Using Cycle Variability Analysis of Cardiac and Photoplethysmographic Signals,” which is incorporated by reference herein in its entirety. A feature CV_ratio refers to a cycle variability score calculated between signals 1 and 3. A feature CV_relative refers to the ratio of the signal 1 to the three signals.

TABLE 2H

| | |
|---|---|
| cv_ovg_CV_ratio_orth13 | cv_ovg_CV_relative_orth1 |

Wavelet Features. The eighth feature set, per Tables 2I and 2J, is directed to features extracted using a wavelet analysis of the signal, specifically the ventricular depolarization, ventricular repolarization, and atrial depolarization. For PPG signals, features extracted using a wavelet analysis of the PPG signal, specifically the systolic and diastolic waveform.

Example descriptions of the features may be found in U.S. Patent Application no. US2023-0072281A1, entitled “Methods and Systems for Engineering Wavelet-Based Features From Biophysical Signals for Use in Characterizing Physiological Systems,” which is incorporated by reference herein in its entirety. A feature Pwave_frequencyRangemedian refers to a median frequency range of a segmented P waveform. A feature QRSwave_timeRangemedian refers to a median time range for a segmented QRS waveform. A feature circularitymedian refers to a median value of circularity for a segmented QRS waveform. A feature cohcircularity refers to circularity of a coherence analysis of a spectral-image feature. A feature dist_peakWidthPSD is the width of an assessed largest peak (at a halfway height of that peak) in a power spectral density function of a high-spectral region of the waveform of interest in a biophysical signal, the power spectral density determined from the high spectral energy region in a binarized spectral image or data. A feature dist_skewPSD refers to the skewness of the power spectral density function of a high-spectral region of the waveform of interest in a biophysical signal, the power spectral density determined from the high spectral energy region in a binarized spectral image or data.

TABLE 2I

| | |
|---|---|
| wt_ovg_Pwave_frequencyRangemedian_orth | wt_ovg_QRSwave_eccentricityiqr_orth1 |
| wt_ovg_QRSwave_timeRangemedian_orth1 | wt_ovg_CohpowerCentroid_orth13 |
| wt_ovg_PwaveDist_stdPSDnanmedian_orth3 | wt_ovg_QRSwave_eccentricityiqr_orth1 |
| wt_ovg_Pwave_orientationiqr_orth3 | wt_ovg_Twave_timeRangemedian_orth3 |
| wt_ovg_Pwave_surfaceAreaiqr_orth1 | wt_ovg_Pwave_timeRangemedian_orth3 |
| wt_ovg_QRSwave_circularitymedian_orth1 | wt_ovg_Cohcircularity_orth23 |
| wt_ovg_QRSwaveDecay_eccentricity_Decaymedian_orth1 | wt_ovg_QRSwaveDist_peakWidthPSDnanmedian_orth1 |
| wt_ovg_TwaveDecay_eccentricity_Decaymedian_orth2 | wt_ovg_QRSwaveDist_qqsse_Normalnanmedian_orth1 |
| wt_ovg_TwaveDecay_timeRange_Decaymedian_orth | wt_ovg_QRSwaveDist_peakDispPSDnanmedian_orth3 |
| wt_ovg_TwaveDist_peakWidthPSDnanmedian_orth3 | wt_ovg_PwaveDist_pdfKernel_L1normnanmedian_orth3 |
| wt_ovg_PwaveDist_peakWidthPSDnanmedian_orth | wt_ovg_TwaveDecay_powerCentroid_Decaymedian_orth1 |
| wt_ovg_QRSwave_frequencyRangemedian_orth1 | wt_ovg_QRSwave_eccentricitymedian_orth1 |
| wt_ovg_QRSwaveDist_entropynanmedian_orth1 | wt_ovg_Twave_frequencyCentroidmedian_orth3 |
| wt_ovg_QRSwaveDecay_timeRange_Decaymedian_orth2 | wt_ovg_Pwave_frequencyCentroidmedian_orth3 |
| wt_ovg_QRSwaveDecay_frequencyCentroid_Decaymedian_orth3 | wt_ovg_QRSwaveDist_peakDispPSDnanmedian_orth1 |
| wt_ovg_QRSwaveDist_qqslop_Normalnanmedian_orth1 | wt_ovg_PwaveDecay_timeRange_Decaymedian_orth3 |
| wt_ovg_PwaveDist_entropynanmedian_orth3 | wt_ovg_PwaveDist_meanPSDnanmedian_orth3 |
| wt_ovg_PwaveDist_pdftKernel_stdnanmedian_orth3 | wt_ovg_QRSwaveDist_skewPSDnanmedian_orth3 |
| wt_ovg_PwaveDecay_orientation_Decaymedian_orth2 | wt_ovg_TwaveDist_pdftKernel_stdnanmedian_orth3 |
| wt_ovg_Twave_frequencyCentroidmedian_orth2 | |

TABLE 2J

| | |
|---|---|
| wt_ppg_Dist_peakWidthPSD_nanmedian_upper_lower | wt_ppg_extent_iqr_upper_lower |
| wt_ppg_Dist_pdftKernel_dpowerPeak_nanmedian_upper_lower | wt_ppg_timeRange_Decay_nanmedian_upper_lower |
| wt_ppg_vpg_eccentricity_nanmedian_upper_lower | wt_ppg_vpg_timeCentroid_Decay_nanmedian_upper_lower |
| wt_ppg_Dist_peakDispRate_nanmedian_upper_lower | wt_ppg_Dist_kurtPSD_nanmedian_upper_lower |
| wt_ppg_orientation_nanmedian_upper_lower | wt_ppg_Dist_peakDispPSD_nanmedian_upper_lower |
| wt_ppg_Dist_numpeakPSD_nanmedian_upper_lower | wt_ppg_vpg_Dist_peakDispPSD_nanmedian_upper_lower |
| wt_ppg_Dist_qqsse_Normal_nanmedian_upper_lower | wt_ppg_vpg_Dist_pdftKernel_dpowerPeak_nanmedian_upper_lower |

Depolarization/Repolarization Waveform Features. The ninth feature set, per Tables 2K and 2L, is directed to features to quantify the propagative characteristics of the ventricular depolarization (VD) wave in three-dimensional space. The main wave characteristics are categorized in four groups based on the 1. velocity, 2. trajectory, 3. orbital frequency (3D rotation rate), and 4. planarity of the wave. In addition to VD wave, the wave propagation features are also applied to the bandpass filtered VD waves at various frequency sub-bands.

For repolarization, similar to Depolarization Wave Propagation, these features are to quantify the propagative characteristics of ventricular repolarization (VR) wave in three-dimensional space. The main wave characteristics are categorized in four groups based on the 1. velocity, 2. trajectory, 3. orbital frequency (3D rotation rate), and 4. planarity of the wave. In addition to VR wave, the wave propagation features are also applied to the bandpass filtered VR waves at various frequency sub-bands.

Example descriptions of the features may be found in U.S. Patent Application no. US2023/0075634A1, entitled “Methods and Systems for Engineering Conduction Deviation Features From Biophysical Signals for Use in Characterizing Physiological Systems,” which is incorporated by reference herein in its entirety.

TABLE 2K

| | |
|---|---|
| repo_ovg_tOffsetAngles_orth1 | repo_ovg_femaleCluster_Cluster5_{—} |
| | orth123 |
| repo_ovg_tOrientation_orth1 | repo_ovg_tOnOffRatio_Median_{—} |
| | orth3 |
| repo_ovg_TpTe_Median_orth2 | repo_ovg_tOffsetAngles_orth2 |
| repo_ovg_tOffsetAngles_orth3 | repo_ovg_femaleClusterDist_{—} |
| | Cluster5_orth123 |
| repo_ovg_femaleClusterDist_{—} | repo_ovg_femaleClusterDist_{—} |
| Cluster1_orth123 | Cluster6_orth123 |

TABLE 2L

| | |
|---|---|
| dwp_ovg_QRS_median_Curvature_sub3_orth123 | dwp_ovg_QRS_minMaxRatio_Planarity_sub4_orth123 |
| dwp_ovg_QRS_max_NormalizedSpeed_sub3_orth123 | dwp_ovg_QRS_minMaxRatio_NormalizedSpeed_sub15_orth123 |
| dwp_ovg_QRS_max_NormalizedSpeed_sub4_orth123 | dwp_ovg_QRS_max_Curvature_sub3_orth123 |
| dwp_ovg_QRS_max_NormalizedSpeed_sub5_orth123 | dwp_ovg_QRS_min_NormalizedSpeed_sub5_orth123 |
| dwp_ovg_QRS_min_Curvature_sub8_orth123 | rwp_ovg_T_max_NormalizedSpeed_sub13_orth123 |
| dwp_ovg_QRS_median_NormalizedSpeed_sub11_orth123 | rwp_ovg_T_median_NormalizedSpeed_sub15_orth123 |
| dwp_ovg_QRS_max_NormalizedSpeed_sub16_orth123 | rwp_ovg_T_median_Curvature_sub14_orth123 |
| dwp_ovg_QRS_min_Curvature_sub16_orth123 | rwp_ovg_T_median_Planarity_sub6_orth123 |
| dwp_ovg_QRS_max_Curvature_sub7_orth123 | rwp_ovg_T_median_NormalizedSpeed_sub13_orth123 |
| dwp_ovg_QRS_min_NormalizedSpeed_sub3_orth123 | rwp_ovg_T_minMaxRatio_Curvature_sub9_orth123 |
| dwp_ovg_QRS_max_NormalizedSpeed_sub15_orth123 | rwp_ovg_T_median_NormalizedSpeed_sub14_orth123 |
| dwp_ovg_QRS_median_NormalizedSpeed_sub8_orth123 | rwp_ovg_T_min_Planarity_sub8_orth123 |
| dwp_ovg_QRS_median_OrbitalFrequency_sub4_orth123 | rwp_ovg_T_median_Curvature_sub10_orth123 |
| dwp_ovg_QRS_max_NormalizedSpeed_sub12_orth123 | rwp_ovg_T_max_Curvature_sub7_orth123 |
| dwp_ovg_QRS_median_Curvature_sub6_orth123 | rwp_ovg_T_max_Curvature_sub3_orth123 |
| dwp_ovg_QRS_min_NormalizedSpeed_sub4_orth123 | rwp_ovg_T_median_NormalizedSpeed_sub7_orth123 |
| dwp_ovg_QRS_min_Curvature_sub4_orth123 | rwp_ovg_T_median_Curvature_sub9_orth123 |
| dwp_ovg_QRS_max_Planarity_sub4_orth123 | dwp_ovg_QRS_minMaxRatio_Planarity_sub4_orth123 |
| wt_ovg_PwaveDecay_surfaceArea_Decaymedian_orth3 | wt_ovg_PwaveDecay_numRegionsR2_Decaymedian_orth2 |
| wt_ovg_TwaveDecay_eccentricity_Decaymedian_orth3 | |

Linear Features. The tenth feature set, per Tables 2M and 2N, is directed to features that quantify both conventional cardiac measurements and variations, using the three biopotential channels, such as waveform amplitudes, durations, heart rate, and morphologies. For PPG signals, features included those to quantify properties of the PPG, as well as a velocity photoplethysmogram (1st time derivative of the PPG; “VPG”) and an acceleration photoplethysmogram (2nd time derivative of the PPG; “APG”), repeated for the red and infrared components of the PPG. Features are calculated using peak amplitudes, peak-to-peak distances, angles between points, and various ratios.

Example descriptions of the features may be found in U.S. Patent Application no. US2023/0055617A1, entitled “Methods and Systems for Engineering photoplethysmographic Waveform Features for Use in Characterizing Physiological Systems,” which is incorporated by reference herein in its entirety.

TABLE 2M
linear_ovg_median_ventricularRepolarizationPeakAmplitude_orth1
linear_ovg_median_septalDepolarizationPeakAmplitude_orth3
linear_ovg_median_repolarizationPotential_orth2
linear_ovg_median_ventricularDepolarizationPeakAmplitude_orth2
linear_ovg_std_septalDepolarizationPeakAmplitude_orth1
linear_ovg_median_repolarizationDuration_orth2
linear_ppg_max_diasPeak_lower
linear_ppg_min_diasPeak_lower
linear_ppg_median_rDenominator_num_upper_lower
linear_ppg_minMax_diasPeak_lower
linear_ppg_mean_diasPeak_lower
linear_ppg_std_systPeak_upper
linear_ppg_VPG_mean_minDist_left_upper
linear_ppg_VPG_std_base_lower

TABLE 2N

| | |
|---|---|
| lvh_ovg_Rpeak2offsetDuration_{—} | lvh_ovg_areaRatio_V3 |
| II | |
| lvh_ovg_RpeakSpeakDistance_{—} | lvh_ovg_onset2RpeakDuration_V6 |
| V1 | |
| lvh_ovg_RpeakArea_V3 | |

Power Spectral. The eleventh feature set, per Table 2O, is directed to features to quantify the power spectrum and frequency content of the PPG or cardiac signals. These features are designed to study the PPG in the frequency domain by obtaining the power spectrum and coherence (cross-spectral analysis).

Example descriptions of the features may be found in U.S. Patent Application no. US2023/0075570A1, entitled “Methods and Systems for Engineering Power Spectral Features From Biophysical Signals for Use in Characterizing Physiological Systems,” which is incorporated by reference herein in its entirety. A feature entropy_coherence refers to an entropy of coherence distribution between two biophysical signals. A feature sum_coherence refers to a cumulative coherence (magnitude squared coherence) between two biophysical signals.

TABLE 2O

| | |
|---|---|
| lvh_ovg_onset2RpeakDuration_{—} | psd_ovg_coherence_entropy_{—} |
| V6 | orth12 |
| psd_ovg_ssr_orth12 | psd_ovg_coherence_sum_signal_{—} |
| | orth123 |

### Example Visualization Output of a Clinical Evaluation System

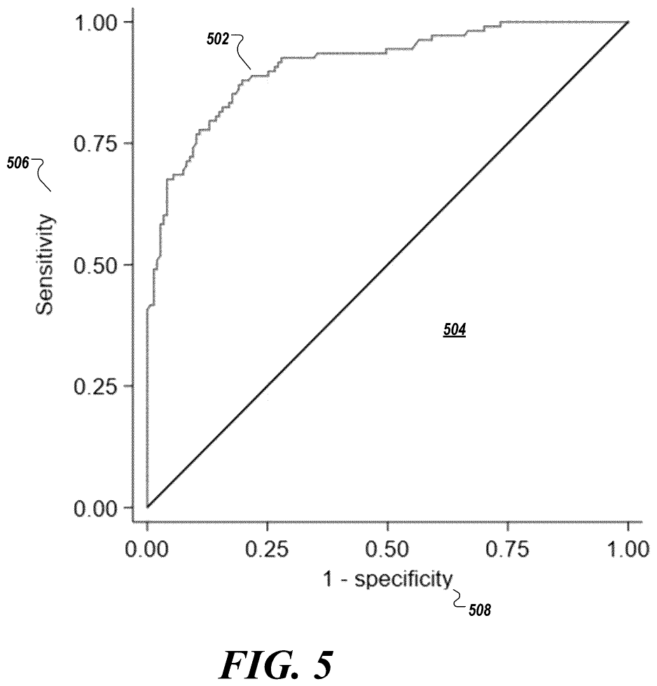

Clinical evaluation system 103a can generate in certain implementations an estimate of the presence or non-presence of elevated PCWP (e.g., the likelihood that the patient has an elevated PCWP). FIG. 4 shows an example patient report 402 that includes a visualization 136 (shown as 136a) of an output score 118 (shown as 118a, 118b, and 118c) generated by the classifier model (e.g., 134) of a clinical evaluation system 103 (shown as 103a).

Clinical evaluation system 103a may include or can operate with a healthcare provider portal 813 (not shown-see FIG. 8) configured to present the patient's report 402 in a user interface. In some embodiments, the healthcare provider portal (e.g., 813) employs a web-service application to provide the patient's report 402 to a remote terminal that is accessible by a healthcare provider, a lab technician, the patient, or the patient's family. In other embodiments, report 402 is transmitted to the biophysical signal capture system (e.g., 102), a patient-owned device, or other display system described herein. To this end, the user interface can be a graphical user interface that can be executed on a desktop computer, tablet, mobile device (e.g., smartphone, smartwatch), measurement device, etc.

Report 402 may present the output score (e.g., 118) in multiple views in the user interface. In the example shown in FIG. 4, the report 402 includes a score summary section 404 (shown as “Patient PCWP Score Summary” section 404), a threshold section 406 (shown as “PCWP Threshold Statistics” section 406), and a frequency distribution section 408 (shown as “Frequency Distribution” section 408). Report 402 also presents other patient information 412 (e.g., identifier, date of birth or age, gender), service provider information 414 (e.g., acquisition location, technician or physician performing the acquisition), and acquisition information 416 (e.g., test date, medical record number). In each of the sections (e.g., 402, 404, 406), Report 402 may include buttons or inputs 418 (shown as 418a, 418b, 418c) that provide panel-specific information and explanation.

Report 402 may indicate a disease-specific result (e.g., elevated PCWP) being available if the signal analysis could be performed. The patient's estimated score (shown via visual element 602a) may be interpreted relative to an established threshold 410 (shown as 410a, 410b, 410c) in one more visualization. In the example of FIG. 4, the score 118 is an estimation that provides a likelihood of the presence of a disease, condition, or an indication of either one (e.g., elevated PCWP) and can be established relative to a validation population in the assessment of that disease, condition, or indication, e.g., using a binary search. If the patient score matches the validation population, that value is presented. If the patient score does not exist within the population, then the location of the closest available score would be used. For example, if the score is 0.55, and the validation population includes scores of 0.52, 0.54, 0.57, then the location of 0.54 may be presented and used to calculate the percentile.

In the score summary section 404 shown in the example of FIG. 4, the patient's score (118a) and associated threshold (410a) is superimposed on a two-tone color bar with the threshold located at the center of the bar with a defined value of “0” representing the delineation between test positive and test negative. The left of the threshold (bar section 420) may be shaded with a lighter color and indicates a negative test result (e.g., “Not Likely Elevated PCWP”), while to the right of the threshold (bar section 422) may be shaded a darker color and indicates a positive test result (e.g., “Likely Elevated PCWP”). The score 118a may include a visual graph indicator (shown as 118a) and a numerical indicator (shown as 118a′). The threshold value, in this implementation, is constant and does not change patient-to-patient.

The threshold section 406 shows reported statistics of the threshold as provided to a validation population that defines the sensitivity and specificity for the estimation. The threshold is the same for every test regardless of the individual patient's score 118, meaning that every score, positive or negative, may be interpreted for accuracy in view of the provided sensitivity and specificity information. The score may change for a given disease-specific analysis as well with the updating of the clinical evaluation.

The frequency distribution section 408 illustrates the distribution of all patients in two validation populations (e.g., (i) a non-elevated population to indicate the likelihood of a false positive estimation and (ii) an elevated population to indicate a likelihood of a false negative estimation). The graphs (424, 426) are presented as smooth histograms to provide context for interpreting the patient's score 118 (e.g., 118b, 118c) relative to the test performance validation population patients.

The frequency distribution section 408 includes a first graph 424 (shown as “Non-Elevated PCWP Population” 424) that shows the score (118b), indicating the likelihood of the non-presence of the disease, condition, or indication, within a distribution of a validation population having non-presence of that disease, condition, or indication and a second graph 426 (shown as “Elevated PCWP Population” 426) that shows the store (118c), indicates the likelihood of the presence of the disease, condition, or indication, within a distribution of validation population having the presence of that disease, condition, or indication.

In the example of the assessment of elevated LVEDP, the first graph 424 shows a non-elevated PCWP distribution of the validation population that identifies the true negative (TN) and false positive (FP) areas. The score 118b is shown in graph 424 along with the threshold 410b. In this same example, the second graph 426 shows an elevated PCWP distribution of the validation population that identifies the false negative (TN) and true positive (FP) areas. The score 118c is shown in graph 426 along with the threshold 410c.

The frequency distribution section 408 also includes interpretative text 428 of the patient's score relative to other patients in a validation population group (as a percentage). In this example, the patient has an PCWP score of −0.08, which is located to the left side of the PCWP threshold, indicating that the patient has “Not Likely Elevated PCWP.”

The report may be presented in the healthcare portal, e.g., to be used by a physician or healthcare provider in their diagnosis for indications of left-heart failure. The indications include, in some embodiments, a probability or a severity score for the presence of elevated PCWP. In other embodiment, the indications include a probability or a severity score for the presence of a condition that could lead to left-heart failure or a likelihood that left-heart failure can result within a certain time frame.

The clinical evaluation system may generate a report for a patient only if a given patient's acquired signal data set meets the signal quality standards as assessed by the signal quality assessment function described herein. The healthcare provider portal may indicate the patient signal acquisition failing to meet signal requirements for a report to be generated. In other embodiments, the healthcare provider portal may not provide an indication that a failed acquisition was made. In some embodiments, such a report may be provided even if the signal quality standards are not met.

### LVEDP and PCWP

LVEDP and PCWP are measures of left ventricular filling pressure. LVEDP corresponds to the instantaneous pressure in the left ventricle immediately before isovolumic contraction and is a metric of left ventricular (LV) compliance, reflecting the rise in LV diastolic pressure following diastolic filling. PCWP is a surrogate for left atrial pressures measured either as a mean value (which does capture the entirety of the cycle) or as an end-expiratory value (a particular time point within the cycle) and is reflective of mean LV filling pressures rather than the instantaneous LV end-diastolic pressure. PCWP can be viewed as a clinical surrogate for LVEDP, and they can be used interchangeably in clinical practice. Invasive assessment of left-sided filling pressures may be performed using either right-heart catheterization (RHC) with measurement of pulmonary capillary wedge pressure (PCWP) or left-heart catheterization (LHC) with direct measurement of left ventricular end-diastolic pressure (LVEDP). PCWP is obtained via venous access without arterial puncture, aortic manipulation, or direct left ventricular instrumentation, whereas LVEDP measurement requires arterial access and catheterization across the aortic valve into the left ventricle. For this reason, PCWP is commonly used in clinical practice to estimate left ventricular filling pressures, reflecting procedural practicality and avoidance of LV instrumentation rather than established superiority in measurement accuracy.

Similarly, [24] reported that the changes in PCWP can be used as a surrogate for LVEDP during resting and exertional stages, as the severity of HFpEF progresses. [21] notes that LVEDP and PAWP (synonym for PCWP) can be employed for diagnosing HFpEF (referred to there as diastolic dysfunction).

Wattanachayakul et al. [24] explicitly characterize PAWP as serving as a surrogate for LVEDP, and report that PAWP correlates well with LVEDP at rest and during exercise in populations suspected of HFpEF. Procedurally, PCWP is obtained via venous access without arterial puncture or direct left ventricular instrumentation, whereas LVEDP measurement requires arterial access and catheterization into the left ventricle; accordingly, PCWP is commonly used in clinical practice to estimate left ventricular filling pressures because it is less invasive and more practical to obtain.

### PCWP and HFpEF

As noted in [22], from a pathophysiological standpoint, HFpEF develops when the left ventricle (LV) is unable to accept an adequate volume of blood during diastole, at normal diastolic pressures, and at volumes sufficient to maintain an appropriate stroke volume [23]. That is, HFpEF develops when the left ventricle (LV) is unable to accommodate an adequate volume of blood during diastole without an abnormal rise in filling pressures, resulting in impaired ventricular filling despite preserved systolic function. These abnormalities are caused by a decrease in ventricular relaxation and/or an increase in ventricular stiffness. And, as reported in [22], the clinical diagnosis of HFpEF remains challenging due to its heterogeneous aetiology.

In being able to determine the left ventricular filling pressure, the exemplary system and method for determining elevated or abnormal PCWP can be used to provide an indication of HFpEF.

Pulmonary artery wedge pressure (PAWP) and pulmonary capillary wedge pressure (PCWP) refer to the same wedge-pressure measurement. von Haehling et al. [22] note that PCWP via right-heart catheterization may be considered for invasive confirmation of HFpEF. Oh et al. [21] summarize guideline-based HFpEF diagnostic criteria that provide invasive hemodynamic cutpoints using either left ventricular end-diastolic pressure (LVEDP) or PAWP/PCWP to define elevated filling pressures.

### EXPERIMENTAL RESULTS AND ADDITIONAL EXAMPLES

Several development studies have been conducted to develop feature sets, and in turn, algorithms that can be used to estimate the presence or non-presence, severity, or localization of disease, medical condition, or an indication of either. In one study, algorithms were developed for the non-invasive assessment of abnormal or elevated PCWP, including those described herein. As noted above, abnormal or elevated PCWP is an indicator of heart failure in its various forms. In another development study, algorithms and features were developed for the non-invasive assessment of coronary artery disease.

The study provides an interpretable, machine-learned algorithm that estimates pulmonary capillary wedge pressure (PCWP) elevation status using hand-engineered features derived from non-invasive physiological signals acquired with a point-of-care device. The population used for sensitivity analysis comprised subjects with elevated PCWP (>18 mmHg) confirmed by right heart catheterization, while the population for specificity analysis included subjects with a low likelihood of PCWP elevation based on guideline-directed echocardiographic criteria.

Four studies contributed to the development activities for PCWP Algorithm as shown in Table 3A below: IDENTIFY Group 4, CADHEALTH, IDENTIFY-PH and RADPH.

The signal data was acquired by the CorVista Capture device, which collects orthogonal voltage gradient (OVG) and photoplethysmogram (PPG) collected simultaneously. For development, capture signals were paired with the outcome of the clinical reference test conducted within each clinical study (see descriptions following) and other clinical metadata. Importantly, all subject data that has been allocated for development purposes (as described below) is excluded from final (blinded) performance validation.

Clinical Metadata Management. Clinical metadata is managed by Clinical Affairs through a 21 CFR part 11 compliant electronic data capture (EDC) system. Access to the EDC is restricted to Clinical Affairs and clinical study sites only. Specifically, Research does not have access to the EDC, thus data is transferred by configured data exports to support algorithm development.

Signal Data Management. CorVista Capture signal data is stored in a data repository, which is managed by the Product Development team. Signal names and subject meta-data entered into the Capture device are managed by Clinical Affairs. Access to signals made available to Research is coordinated between the Clinical Affairs and Product Development teams.

TABLE 3A

| | | | Function | | |
|---|---|---|---|---|---|
| | | | Dimen- | Training | Additive |
| | | Number | sionality | & | for model |
| | | of | Reduc- | internal | assess- |
| Dataset | Protocol | subjects | tion | validation | ment |
| IDENTIFY | CADF- | 161 | | 161 | |
| Group 4 | CIP-001 | | | | |
| CADHealth | CADF- | 550 | | | 550 |
| Group 1 | CIP-001 | | | | |
| CADHealth | | 108 | | | 108 |
| Group 2 | | | | | |
| IDENTIFY-PH | PHDEV- | 314 | 46 | 122 | |
| Group 1 | CIP-001 | | | | |
| RADPH | RADPH- | 59 | 54 | | 59 |
| | CIP-001 | | | | |

| | | | | |
|---|---|---|---|---|
| TOTAL | 1192 | 100 | 285 | 717 |

IDENTIFY Group includes subjects with new onset symptoms undergoing CTA, and specifically the subgroup who also have a recent echocardiogram available. Disease label is defined as non-diseased when the CADRADS (Coronary Artery Disease Reporting And Data System) (Cury et al., 2016) is less than or equal to 2, corresponding to a maximal lesion <50%, and when the echocardiogram indicates no evidence of diastolic dysfunction nor pulmonary hypertension as determined by a core lab. IDENTIFY-PH Group includes subjects with new onset symptoms referred for RHC. Disease label is assessed based solely on PCWP IDENTIFY-PH. RADPH includes symptomatic subjects who have undergone right catheterization within the prior 18 months, who demonstrated a mPAP of at least 30 mmHg and PCWP measured. Disease label is assessed based solely on PCWP. CADHEALTH Group 1 includes asymptomatic healthy younger subjects with no risk factors, which minimizes the likelihood of undetected underlying elevated PCWP. Disease label is defined as non-diseased based on the inclusion/exclusion criteria. CADHEALTH Group 2 includes asymptomatic older healthy subjects with no risk factors, which minimizes the likelihood of undetected underlying elevated PCWP. Disease label includes non-diseased based on the inclusion/exclusion criteria.

Dimensionality Reduction Using Feature Selection. Given the large feature library, particularly as compared to the number of subjects, a dimensionality reduction step to reduce the number of features was undertaken using univariate feature selection. Prior to entering machine learning, each feature was assessed individually for statistical ability to separate diseased subjects from non-diseased subjects. Statistical testing was performed on an N=100 dataset, comprised of an equal division into diseased and non-diseased. The N=50 diseased subjects had PCWP>18 mmHg, defined as elevated per the rational used in PCWP-UR-001. The N=50 non-diseased subjects had PCWP≤18 mmHg, chosen to reduce the effect size, and thus increase the predictivity of the selected features. Care was taken at this stage (which carries through the process) to ensure that the genders were treated evenly; in this case, the number of males and females in each of the diseased and non-diseased groups were equal. The subject IDs used can be found in Attachment 1. The univariate tests used to determine if the feature significantly separated the two cohorts were t-test (to detect a difference in the means of the distributions), area under the receiver operator characteristic curve (ROC-AUC) (treating the feature as a predictor of the disease state) and mutual information (to detect differences in the shapes of the distributions).

To select features using t-test, a threshold on the p-value was established at 0.0075 (much lower than the conventional threshold of 0.05). To select features used ROC-AUC, bootstrap sampling was used to calculate the 95% confidence interval, the lower bound of which must be greater than 0.5375, or the upper confidence bound must be less than 0.4625. To select features using mutual information, bootstrap sampling was used to calculate the 95% confidence interval, the lower bound of which must be greater than 1.2. To remove features with small means and small variations (to reduce sensitivity to computational precision), the mean and standard deviation of the feature must be greater than 0.001. Finally, we remove highly correlated features with a threshold of 0.95 to minimize redundancy and improve model generalization. This helps enhance feature diversity and ensures the model focuses on more informative signals.

The study was conducted in two stages: development and validation. Subjects for each stage were consecutively enrolled across 19 U.S. clinical sites and separated based on enrollment date. During development, the algorithm was trained on subjects enrolled prior to a pre-specified cutoff date, with co-primary performance goals of sensitivity >70% and specificity >60%. The validation stage used subjects enrolled after the cutoff date, and yielded a sensitivity of 82.4% (95% CI: 75.2-89.6%) and specificity of 83.0% (95% CI: 76.9-89.1%), exceeding the pre-specified targets. The ROC-AUC was 0.912, and performance remained consistent across key subgroups including sex, age, and race. The study notes three distinct methodological advantages. First, the validation design employed enrollment-date separation—widely regarded as the gold standard for algorithm validation. Second, the cohort was not limited by HF type, and consisted of both HFrEF and HFpEF patients (a group in whom diagnosis is often more challenging due to the absence of EF reduction). Third, the model was based on hand-engineered features, allowing for greater model interpretability. The algorithm is currently under review at the FDA for future commercial usage. We believe this work represents a meaningful and timely contribution to the growing interest in non-invasive hemodynamic assessment for heart failure.

PCWP discussion. Pulmonary capillary wedge pressure (PCWP) is a beneficial hemodynamic parameter with broad utility in evaluating cardiovascular disease. Elevated PCWP is associated with a range of conditions, including heart failure (HF), hypertrophic cardiomyopathy, and valvular heart disease, and helps differentiate between cardiogenic and non-cardiogenic pulmonary edema. PCWP also serves to distinguish between pre-capillary and post-capillary pulmonary hypertension (PH), where the treatment pathways diverge significantly.

Despite the clinical benefits of PCWP, direct measurement requires right heart catheterization (RHC), an invasive and resource-intensive procedure typically confined to specialized facilities. As a result, many patients with elevated left ventricular (LV) filling pressures remain undiagnosed or misclassified, delaying appropriate therapy and worsening outcomes. The challenge is particularly acute in the context of HF with preserved ejection fraction (HFpEF), which has a similar prevalence to HF with reduced EF (HFrEF) but remains underdiagnosed due to the limitations of available non-invasive diagnostic tools.

Transthoracic echocardiography (TTE) is currently the most widely used non-invasive alternative to estimate PCWP via surrogate markers of diastolic dysfunction. However, several limitations constrain its diagnostic utility. The sensitivity of TTE for detecting diastolic dysfunction is modest, particularly in patients with preserved EF. In the largest validation study to date, the 2016 ASE/EACVI guideline-based algorithm showed a sensitivity of just 35% for detecting elevated PCWP in patients evaluated for unexplained dyspnea [1], yet at a robust specificity of 90%. Furthermore, up to 61% of patients lack measurable parameters such as tricuspid regurgitant jet velocity, resulting in indeterminate classifications [2]. Challenges related to access are more pronounced in resource-limited settings, where skilled echocardiographers, and even echocardiography systems themselves, may be unavailable.

While it is difficult to establish a precise absolute level of PCWP elevation that is clinically significant, both the pivotal CHAMPION [3] and post-approval GUIDE-HF [4] trials of CardioMEMS, an implantable pulmonary artery pressure sensor, provide important context. In CHAMPION, a single-blind, prospective, randomized trial in HF NYHA Class III (moderately symptomatic) patients, optivolemic was defined as diastolic pulmonary artery pressure (dPAP), (a close surrogate of PCWP) from 8-20 mmHg; above that range, and physicians in the treatment arm of the trial aimed to reduce dPAP to within the optivolemic range by guideline-directed therapy [5]. CHAMPION demonstrated a 37% reduction in HF-related hospitalization in the treatment arm as compared to the control over the entire 15-month follow-up [3]. Critically, CHAMPION also showed similar efficacy in the HFpEF (50% reduction) subgroup [6], implying continued relevance of the optivolemic upper limit of 20 mmHg when EF is preserved. The subsequent GUIDE-HF trial broadened the population and demonstrated efficacy in NYHA Class II (mildly symptomatic) [4].

CHAMPION and GUIDE-HF demonstrated the clinical imperative of knowledge of LV filling pressure in the management of HF; yet, it is not often possible to invasively measure PCWP, particularly in rural and underserved urban areas in the US. Therefore, we aimed to develop and validate a non-invasive, point-of-care test for the detection of elevated PCWP. Based on the optivolemic range used by CHAMPION and GUIDE-HF, which demonstrated broad applicability across HF types (reduced and preserved EF) and symptomology (NYHA Classes II & III), we defined clinically-relevant PCWP elevation at >18 mmHg. PCWP may be overestimated by dPAP, for example in patients with elevated mean pulmonary artery pressure, rendering the >18 mmHg threshold an appropriate adjustment for PCWP to the CHAMPION and GUIDE-HF definition that is reasonable for point of care use. Therefore, the definition of PCWP>18 mmHg as elevated will allow the CorVista system to detect elevated PCWP early in the clinical course of HFrEF, HFpEF and other conditions associated with elevated LV filling pressures.

Within the feature development phases, features were developed, including several of features associated with modules 322, 324, 326, 328, 330, 332, 334, 336, 338, 340, 342, among other features, to extract characteristics in an analytical framework from biopotential signals (as an example of the cardiac signals discussed herein) and photo-absorption signals (as examples of the hemodynamic or photoplethysmographic discussed herein) that are intended to represent properties of the cardiovascular system. Corresponding classifiers were also developed using classifier models, linear models (e.g., Elastic Net), decision tree models (XGB Classifier, random forest models, etc.), support vector machine models, and neural network models to non-invasively estimate the presence of an elevated or abnormal PCWP. Univariate feature selection assessments and cross-validation operations were performed to identify features for use in machine learning models (e.g., classifiers) for the specific disease indication of interest.

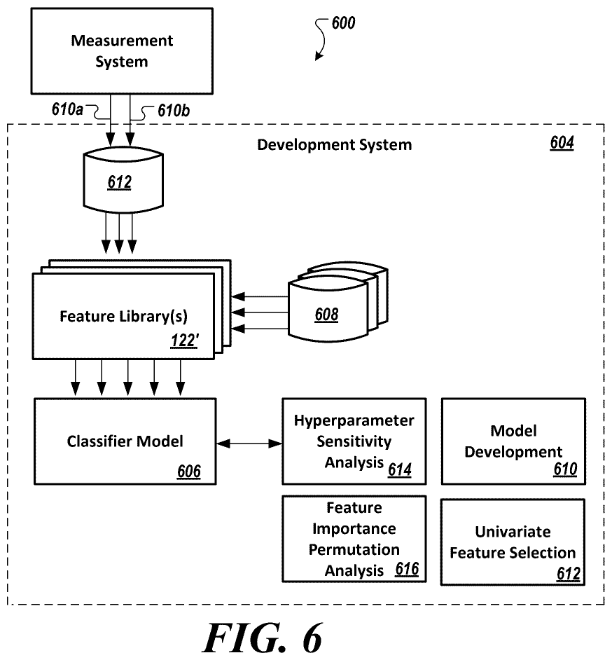

FIG. 5 shows the performance of the classifier (e.g., 134) for the elevated PCWP estimation/assessment algorithm that employs the features of modules 322, 324, 326, 328, 330, 332, 334, 336, 338, 340, 342 in a receiver operating characteristic (ROC) curve (502) of 1-specificity vs. sensitivity. The ROC curve (502) illustrates the diagnostic ability of a classifier system (e.g., 134) as its discrimination threshold is varied. The ROC curve may be created by plotting the true positive rate (TPR) against the false positive rate (FPR) at various threshold settings. AUC-ROC quantifies the area under a receiver operating characteristic (ROC) curve—the larger this area, the more diagnostically useful the model is at accurately performing the classification. The ROC, and AUC-ROC, value is considered statistically significant when the bottom end of the 95% confidence interval is greater than 0.50. In the example ROC curve 502 of FIG. 5, the y-axis (506) (shown as the sensitivity axis (“Sens” 506) can be defined as

$$sens = \frac{TP}{TP + FN}$$

and the axis (208) (shown as “1-Spec” 508) can be defined as

$$1 - spec = \frac{FP}{TN + FP}$$

in which TP is the probability of a true positive, FN is the probability of a false negative, TN is the probability of a true negative, and FP is the probability of a false positive.

In the example of FIG. 5, the algorithm achieved a sensitivity of 82.4% (95% CI: 75.2-89.6%) and specificity of 83.0% (95% CI: 76.9-89.1%) per Table 3B. Therefore, the prospectively set co-primary endpoint was met against the goals of a lower confidence bound of 70% sensitivity (p=0.0005) and 60% specificity (p<0.0001). The ROC-AUC (FIG. 4) was 0.912 (95% CI: 0.891-0.933) among a set of validation data in estimating the likelihood of a patient having elevated PCWP. This specificity and sensitivity performance is comparable to the gold standard techniques for the assessment of elevated PCWP (namely cardiac catheterization), which is a highly invasive assessment method. Notably, the exemplary system and method can provide a similar clinical assessment using non-invasively acquired measurements (e.g., cardiac and PPG signals) that can be acquired from surface-based sensors placed on the patient while at rest. Indeed, the ROC curve 502 (and corresponding AUC 504) shows the elevated PCWP estimation/assessment algorithm disclosed herein having significant utility in the assessment of the presence or non-presence of elevated PCWP.

TABLE 3B

| Co-primary | Point | | | p-value vs |
|---|---|---|---|---|
| Endpoints | Estimate | 95% CI | Goal | goal |
| Sensitivity | 82.4% | (75.2%, | 70% | 0.0005 |
| | (89/108) | 89.6%) | | |
| Specificity | 83.0% | (76.9%, | 60% | <0.0001 |
| | (122/147) | 89.1%) | | |

The threshold can be selected to optimally establish the threshold that maximizes areas of the true positive (FP) and true negative (TN) while minimizing the areas of the false positive (FP), false negative (TN) within the validation populations. The selected threshold value (e.g., 0.39 discussed above) can be subtracted from all the predictions to center the threshold at zero; therefore, any prediction greater than or equal to zero is test-positive, and any prediction less than zero is test-negative.

Study Design and Population. The prospective, multicenter, non-randomized, consecutively enrolled, selectively blinded repository trials enrolled symptomatic patients referred for RHC (IDENTIFY-PH; NCT04031989) or undergoing TTE (IDENTIFY Group 4; NCT03864081), for the purpose of developing and validating machine-learned algorithms. Non-invasive signals from a proprietary acquisition device were acquired prior to the reference test. Subjects provided written informed consent, and both studies were approved by the Western Institutional Review Board and are available on clinicaltrials.gov. Studies were funded by Analytics for Life. The studies were conducted at 19 clinical sites across the United States. Inclusion criteria required participants to be ≥18 years old with cardiovascular symptoms. Exclusion criteria included prior valve replacement, congenital heart disease, implantable cardiac devices, and conditions interfering with signal acquisition. Supplementary Appendix Section 2 contains the complete study inclusion/exclusion criteria.

Signal Acquisition. As part of these two development studies, clinical data were collected from adult human subjects using a biophysical signal capture system and according to protocols described in relation to FIG. 2. Physiological signals were collected using a proprietary device that records orthogonal voltage gradient (OVG) via seven electrodes on the thorax, and photoplethysmogram (PPG) signals via a fingerclip sensor. Patients were supine and at rest during the 210-second recording. Signals were sampled at 8 kHz and paired with patient demographics. Signals were evaluated using a signal quality assessment (SQA) tool, including assessment of high frequency and powerline noise [8]; only signals that passed were included in the development and validation datasets.

Cohorts. PCWP measurements were obtained in IDENTIFY-PH from gold standard RHC performed within seven days of signal acquisition. Subjects with elevated PCWP>18 mmHg comprised the positive cohort (Population A). Population B, the symptomatic negative cohort, was composed of subjects from IDENTIFY Group 4 who had a recent negative coronary computed tomographic angiography (CCTA), showing a CADRADS score from 0-2, with no recommendation for additional testing. Population B subjects also demonstrated low probability of elevated PCWP, based on the following TTE criteria: low probability of PH per the ESC/ERS guidelines [9], and absence of diastolic dysfunction per the ASE guidelines [10], both as evaluated by an independent core laboratory (Global Institute for Research, Midlothian, VA).

Algorithm Development. The algorithm development followed a similar process to that used for the development of previous algorithms for obstructive coronary artery disease and pulmonary hypertension, both of which were developed [11], [12] and validated [13], [14] using signals collected using the same acquisition device.

The first stage was feature selection; a large library of physiological measurements has previously been developed and described, which was reduced using univariate feature selection on a non-overlapping group of N=100 subjects, to 243 features. Subsequently, the training cohort was assembled, composed of N=122 Population A subjects and N=161 Population B subjects (Supplementary Appendix Section 4). Machine learning was then used to train to predict the Population (A or B) using a continuous score on the training dataset of N=283, using the N=243 features. The machine learning algorithm was a bagged ensemble of 500 CatBoost models, a gradient boosting algorithm well suited for small datasets.

Out-of-fold (OOF) prediction was used to enable detailed analysis of the resultant model in the training cohort in a robust, naïve manner. The initial step of the OOF analysis was the cut-point selection, i.e., the delineation point that defines test-positivity vs negativity, which was chosen to result in a model sensitivity of 90%, and specificity of 76%. The cut-point was subtracted from the model output, to result in an effective test-positivity definition of >0. Subsequently, the OOF performance was assessed for differences across the critical subgroups of gender, age (>65) and BMI (≥30), and no statistically significant differences were found. Further, in the subset of the Population A cohort with the EF measured (56% of all Population A subjects had an EF available), there was no significant difference in the sensitivity in the group with EF≤40% (N=23, sensitivity of 96%) and EF>40% (N=45, sensitivity of 91%), with p=0.85. However, interestingly, the test score in the EF≤40% was significantly higher than EF>40% (p=0.002). Finally, a stability analysis on OOF was performed (Supplementary Appendix Section 7), which supported the reliability of the model output.

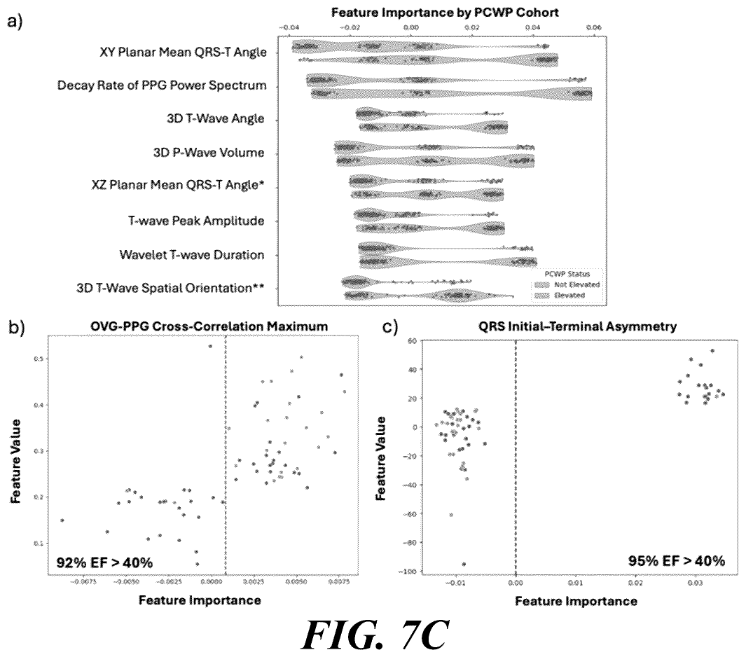

The importance of the features to the model output was characterized by LIME (Local Interpretable Model Explanations) for subjects with elevated and non-elevated PCWP individually (FIG. 7A, subpanel a), which illustrates the importance of phase space geometry of the OVG signal (e.g., spatial angles and volumes) in determining both test positivity and negativity. Specifically, three-dimensional P-wave volume was found to be critical feature; unsurprising, given the impact on atrial conduction from elevated pressures. Further, T-wave amplitude and duration were top-ranked, illustrating the importance of repolarization abnormalities on the algorithm output. FIG. 7C, subpanel b and c are visualizations of features found to differ significantly across EF subgroups, ≤40% (HFrEF) and >40% (HFmrEF and HFpEF), using t-test with Bonferroni correction across all algorithm input features. FIG. 7C, subpanel b shows the relationship between feature importance and value of the OVG-PPG Cross-Correlation Maximum, a measurement of the relationship between the two signal modalities. The feature value positively correlates with the importance, showing that higher feature values result in higher model outputs; thus, implying that a closer relationship between the modalities is more indicative of LV filling pressure elevation. Further, the line segregating the plot shows a rudimentary clustering of HF types, with EF>40% tending to have lower feature values, and thus a weaker relationship between the modalities. FIG. 7C, subpanel c visualizes QRS Initial-Terminal Asymmetry, with positive values reflecting a shorter terminal portion than initial and vice versa for negative values. Interestingly, values greater than approximately 20 ms (“positively” asymmetric) increased the resultant score, representing a group almost entirely composed of subjects with EF>40% (95%), whereas negative score influence was typically demonstrated by feature values close to zero (symmetric), and composed of a mix of EF types. The relationship between these feature values, importances, and EF subgroups suggests that LIME may have utility beyond understanding how the score value was reached, and specifically, may enable phenotyping of HF type (preserved vs. reduced EF), and will be explored in future work.

The development of the model was concluded by locking the software, including the cut-point, and embedding it in a high-throughput processing system to enable evaluation on the blinded validation dataset.

Model Types. The study used capped CatBoost in PCWP Algorithm. CatBoost (Categorical Boosting) is a gradient boosting algorithm developed by Yandex, designed to efficiently handle both categorical and numerical features in machine learning tasks. It is widely used for classification, regression, ranking, and time-series forecasting. One of the key advantages of CatBoost is its ability to process categorical data without requiring extensive preprocessing. Unlike other boosting algorithms such as XGBoost and LightGBM, which often require label encoding or one-hot encoding for categorical variables, CatBoost implements ordered target statistics and one-hot encoding to handle categorical features directly (Dorogush et al., 2018). Additionally, CatBoost utilizes oblivious trees, a specialized symmetric decision tree structure that ensures balanced splits at each level. This design helps reduce overfitting, improve generalization, and increase prediction speed compared to traditional asymmetric trees (Prokhorenkova et al., 2018).

CatBoost is optimized for computational efficiency and can leverage GPU acceleration, making it one of the fastest gradient boosting frameworks available. It is particularly effective for small datasets where other boosting models may struggle with overfitting (Prokhorenkova et al., 2018). The CatBoost implementation has been extended to include a capping procedure, as CatBoost can produce probability estimates that slightly exceed the range [0,1]. Given that the intended output is a probability score between 0 (non-diseased) and 1 (diseased), the presence of values outside this range is undesirable.

Without capping, CatBoost may generate predictions such as −0.1, 1.2, or even more extreme values, which could compromise both interpretability and downstream usability. This issue is particularly relevant when visualizing probability distributions, for example, in Kernel Density Estimation (KDE) plots. A single extreme value, such as a prediction significantly greater than 1, can distort the entire distribution, leading to misleading representations. Moreover, many post-processing steps, including threshold-based classification, ensembling, and risk stratification, assume probability values are strictly bounded within the [0,1] range. To address this, the capping procedure ensures that any output value greater than 1 is set to 1, and any output value less than 0 is set to 0. This adjustment stabilizes the predictions, prevents unintended distortions in visualization, and ensures that probability estimates remain valid and reliable for clinical decision-making.

Hyperparameters of CatBoost, which modify the behaviour of the algorithm, include: (i) Boosting type: Ordered Boosting, the default option, helps reduce overfitting, while Plain Boosting follows a more traditional gradient boosting approach, (ii) iterations: Number of boosting rounds or trees trained. Increasing the number of iterations can improve performance but may lead to overfitting if set too high, (iii) L2 Leaf Reg: Controls the L2 regularization applied to leaf values. This helps prevent overfitting by smoothing the model's predictions. Higher values enforce stronger regularization, making the model less sensitive to noise, (iv) Border Count: Determines how many discrete bins are used to convert continuous features into categorical values. A higher border count captures more detail but increases computational complexity, and (v) Bagging Temp: Influences the level of randomness in the bagging process during training. A higher value increases the diversity of trees, helping to reduce overfitting, while a lower value makes training more deterministic.

Table 4 provides an example hyperparameter search employed in the study.

TABLE 4

| Hyperparameter | Searched Values |
|---|---|
| Boosting | Plain, ordered |
| Iterations | 500, 1000, 1500, 2000, 4000, 6000, 8000, 10000 |
| L2 Leaf Reg | 1, 3, 5, 7, 10 |
| Border Count | 32, 64, 128, 254 |
| Bagging Temp | 0.0, 0.33, 0.66, 1.0 |

Algorithm Validation. The algorithm validation was performed on a blinded, enrollment-gated validation dataset composed of the same cohort definitions as development: Population A as the positive group, with PCWP>18 mmHg, and Population B as the negative group, with low probability of PCWP elevation based on TTE evaluation. Subjects cannot be absent from the RHC result in Population A, nor the TTE result in Population B. The validation dataset was chronologically separated from the validation dataset using enrollment date. Given that separation, some of the sites participated uniquely in either development or validation (i.e., not both). The co-primary endpoint was composed of sensitivity, which was evaluated on Population A, and specificity, which was evaluated on Population B. To power the co-primary endpoint at 80%, sensitivity and specificity must each be powered at 90% (90%×90%=81%).

To be an effective front-line test, a large proportion of the subjects that truly have elevated PCWP should be identified, and thus a high sensitivity is beneficial, at the consequence of a lower specificity. Given that the current front-line test, TTE, has a sensitivity of 35% (in the 39% of subjects with measurable TRV) for diastolic dysfunction, a test with a lower confidence bound (LCB) on the sensitivity substantially greater than that would have significant clinical utility; the sensitivity LCB was therefore set to 70%, with α=0.05, as assessed using normal approximation test. The validation sensitivity point performance was estimated to be 82% for powering purposes, requiring a sample size of N=107 to power the sensitivity endpoint at 90%.

Similarly, the specificity of TTE was used in setting the required LCB for the test. While the specificity of TTE is robust at 90%, up to 61% of subjects may be absent the required TRV measurement to complete the guideline assessment for diastolic dysfunction. Therefore, the effective specificity is more accurately stated as 90% (100-61%)=35%. Therefore, a performance goal of specificity LCB of 60% is reasonable, with α=0.05, as it represents an improvement over TTE. The validation specificity point performance was estimated to be 76% for powering purposes, requiring a sample size of N=72 to power the specificity endpoint at 90%.

In addition to the co-primary endpoints of sensitivity and specificity, the area under the receiver operator characteristic curve (ROC-AUC) was evaluated, as well as positive, negative, and instantaneous likelihood ratios. Subgroups were also assessed using sensitivity and specificity, with α=0.01.

The treating physicians did not have access to their patients' test scores, and thus, scores were not considered in patient treatment. Conversely, neither the RHC nor the TTE results were available to those generating the test scores on the validation dataset. The test scores and RHC/TTE results were paired by an independent statistician (MRH Biostatistics, LLC).

### Results

Enrollment into the validation dataset was conducted consecutively from February 2021 to September 2022; given that all development enrollment was completed prior to enrollment into validation, the two datasets are gated by enrollment date—the gold-standard approach for validation. N=149 subjects with a PCWP result were enrolled into Population A (FIG. 3A), of which N=9 had a major protocol deviation (e.g., signal acquisition and catheterization separated by more than 7 days). N=11 did not have a signal received due to not having enough time to perform signal collection prior to the catheterization procedure, or due to connectivity issues or improper use of the device (i.e., not keeping the device properly charged and/or connected to the internet). Finally, N=21 subjects did not have a signal passing SQA, leaving N=108 as the final Population A group for analysis. A total of N=170 subjects with a negative CCTA were enrolled into Population B (FIG. 3B), and N=18 had major protocol deviations. Of those, N=2 did not have any corresponding signal, and N=3 did not have a signal passing SQA. Therefore, the final analysis group for Population B was N=147.

No treatment-emergent adverse events were reported in either Population A or B. All subjects in Population B and 97.3% in Population A had symptoms, with the most common being dyspnea during exercise or exertion (65.3%), fatigue or decreased capacity to exercise (39.6%), chest pain at rest (43.5%), and dyspnea at rest (28.6%). All other symptoms were reported by 46.6% of subjects and include syncope, dizziness, etc. The mean PCWP in Population A was 23.9±5.0 mmHg, ranging from 19 mmHg to 46 mmHg.

In comparison to Population B, Population A tended to be older and had a higher frequency of males. Racial and BMI distributions were similar, and both populations exhibited a spectrum of cardiovascular risk factors. The only meaningful difference between the demographics of development and validation (Supplementary Appendix Section 8) was a higher prevalence of older subjects in validation. The validation cohort was enrolled across a range of US geographies (Supplementary Appendix Section 9).

The algorithm achieved a sensitivity of 82.4% (95% CI: 75.2-89.6%) and specificity of 83.0% (95% CI: 76.9-89.1%). Therefore, the prospectively set co-primary endpoint was met against the goals of a lower confidence bound of 70% sensitivity (p=0.0005) and 60% specificity (p<0.0001). The ROC-AUC (FIG. 4) was 0.912 (95% CI: 0.891-0.933). No significant differences were observed at the pre-defined α=0.01, including in the critical subgroups of age, sex, and race.

Discussion Positive predictive value (PPV) and negative predictive value (NPV) are dependent on the prevalence of the disease; in the general population, the prevalence of heart failure is thought to be approximately 2% [17]. At that low prevalence, the test has an excellent NPV of 99.6% and a modest PPV of 9.0%. However, the prevalence of heart failure in dyspneic patients is substantially higher, for example, reaching 30% in a community cohort [18]; at that prevalence, the NPV of the device maintains excellence at 91.5%, but the PPV is significantly improved at 67.4%. Clearly, the predictive values of the test are highly dependent on the prevalence in the most relevant population to the patient; therefore, a patient-specific approach, requiring estimating the pre-test probability, can leverage the test's likelihood ratios (LR). The test's positive LR is 4.85, and negative LR is 0.21; therefore, the result is an approximate five-fold increase from the patient's pre-test odds to post-test odds, with a similar reduction with a negative test. For instance, if the physician estimated the pre-test probability at 50%, then a positive test would update the probability of PCWP elevation to 83%; similarly, a negative test would result in an updated probability of 17%. Further, while the test was formally validated in a binary fashion (test-negative/test-positive), there may be utility in the continuous value of the score, such as in instantaneous LR, which provides a LR at each distinct test score value (Supplementary Appendix Section 11). Instantaneous LR is lowest with the most strongly negative test scores, and highest with the most strongly positive test scores. For instance, if that same hypothetical patient with a pre-test probability of 50% has a score of −0.22, that yields an LR of 0.14, resulting in a post-test probability of 12%. Similarly, a score of 0.31 for that patient yields an LR of 7.21, with a post-test probability of 88%.

As discussed, the sensitivity of TTE for detecting diastolic dysfunction is modest-perhaps as low as 35% [1], prior to accounting for patients with immeasurable TRV. Further, TTE is difficult to widely deploy due to challenges in the availability of skilled technicians and devices. AI-ECG algorithms have been developed which aim to predict the presence and stage of diastolic dysfunction, using TTE as the ground truth—while the performance appears effective (e.g., 0.91 AUC [19]), it must be contextualized by the lack of sensitivity of TTE, which propagates into the generation of a potentially biased positive population (i.e., lacking TTE false negative patients). Further, AI-ECG approaches offer only limited insight into the mechanism of prediction generation for any given patient, such as highlighting a region of the ECG that contributed most to the output. In contrast to TTE and AI-ECG approaches, this test was developed using a robust representation cohort (TTE for negatives, and gold-standard RHC for positives), is inherently interpretable with the combination of hand-engineered features and LIME, can be deployed anywhere with an internet connection, and the robust performance (AUC: 0.912; sensitivity: 82%; specificity: 83%) benefits both the physician and patient.

The validation dataset is representative of the true population that may be evaluated using this test based on four critical characteristics. Firstly, the demographics are reflective of the composition of the US; 18.8% of the validation subjects were Black or African American, greater than the 14.4% in the US population [20], which is imperative for a technology involving photoplethysmography, since melanin has been shown to differentially affect the absorption of the light emitted by the sensor [21]. Secondly, the population included 59.2% female subjects. Both women and minority groups have been historically underrepresented in cardiology studies [22]; however, our validation dataset included women in substantial numbers, and no significant differences in performance were observed. Third, enrollment was geographically diverse, with substantial representation from the Southeastern United States, as well as contributions from the Southwest, West, and Northeast regions. Finally, the subjects were symptomatic, which is crucial as the test is only intended to be used on symptomatic patients, and including asymptomatic patients may falsely inflate the specificity [23].

### Example Development System

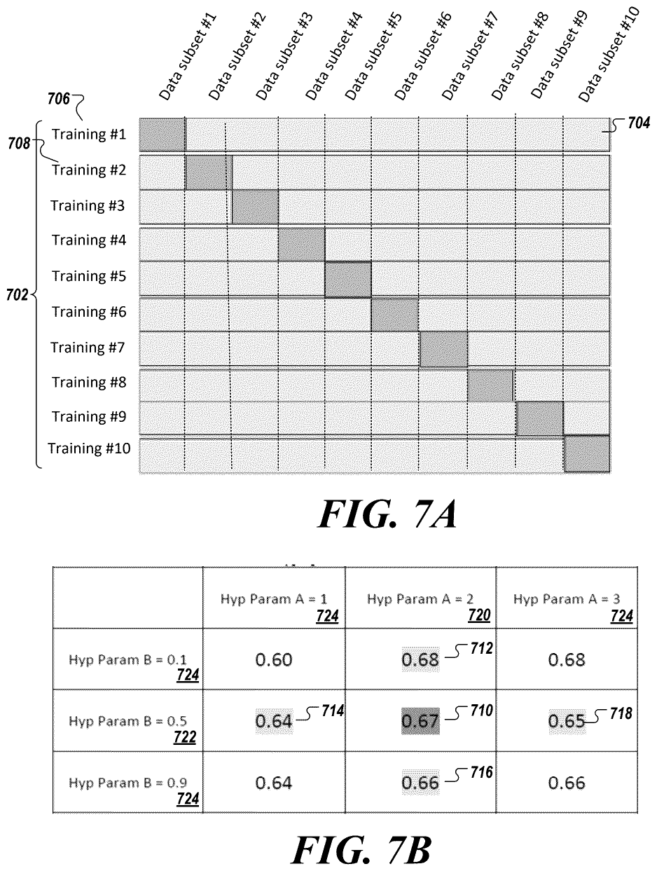

FIG. 6 is a diagram of a development system 600 that can be used to develop and evaluate a clinical assessment system (e.g., system 103) that can estimate (e.g., determine) the likelihood of the presence, non-presence, localization, and/or severity of a metric/estimate (e.g., elevated PCWP) associated with a disease or condition in such a physiological system, including for elevated PCWP as disclosed herein, in accordance with an illustrative embodiment. The development system 600 can be used to assess and develop analytical engines for various clinical applications discussed herein.

Development system 604 can include a set of feature library module(s) (e.g., 122′ and 608), classifier model module(s) 606, training data set repository 612, model development module 610, univariate feature selection module 612, hyperparameter sensitivity analysis module 614, and feature importance permutation analysis 616. Development system 604 may employ one or more of these modules to select features prior to classifier or model development by isolating features that are stable, meaningful, and statistically linked to a patient's disease, medical condition, or indication of either. Features that do not pass through the selection step can be excluded from machine learning for the specific disease, condition, or indication.

Feature Library Module. The feature library module 122 (shown as 122′) can include feature (e.g., from modules 322, 324, 326, 328, 330, 332, 334, 336, 338, 340, 342). Other feature modules may be employed by the development system 604, shown as features modules 608, e.g., including those that are described in U.S. Pat. Nos. 9,289,150; 9,655,536; 9,968,275; 8,923,958; 9,408,543; 9,955,883; 9,737,229; 10,039,468; 9,597,021; 9,968,265; 9,910,964; 10,672,518; 10,566,091; 10,566,092; 10,542,897; 10,362,950; 10,292,596; 10,806,349; U.S. Patent Publication nos. 2020/0335217; 2020/0229724; 2019/0214137; 2018/0249960; 2019/0200893; 2019/0384757; 2020/0211713; 2019/0365265; 2020/0205739; 2020/0205745; 2019/0026430; 2019/0026431; PCT Publication nos. WO2017/033164; WO2017/221221; WO2019/130272; WO2018/158749; WO2019/077414; WO2019/130273; WO2019/244043; WO2020/136569; WO2019/234587; WO2020/136570; WO2020/136571; U.S. patent application Ser. Nos. 16/831,264; 16/831,380; 17/132,869; PCT Application nos. PCT/IB2020/052889; PCT/IB2020/052890, each of which is hereby incorporated by reference herein in its entirety. While the run-time system, e.g., of FIG. 1 may include some or only features used for a given clinical evaluation (e.g., tens to hundreds of features), the feature libraries 608 of the development system 604 can include the full set of features (or a substantial portion) to be evaluated for a given clinical application (e.g., in the hundreds to thousands of features).

Classifier Model Module. The classifier model module 606 can include instructions for a plurality of configured machine learning and regression models, such as, but not limited to, decision trees, random forests, SVMs, neural networks, linear models, Gaussian processes, nearest neighbor, SVMs and Naïve Bayes, including those described in relation to FIG. 1. The classifier model module 606 can include instructions for commercially available machine learning and regression models (e.g., as manufactured by Caret or MathWorks) or custom machine learning and regression models, e.g., configured by the model development module 610 and other development modules described herein (e.g., 614, 614, 616). In some embodiments, the classifier model module(s) 606 is configured with a corresponding configuration operation to tune or configure the machine learning and regression models.

The classifier model module 606 may include machine learning and regression models for the assessment of elevated or abnormal left ventricular end-diastolic pressure (PCWP), as well as various other clinical applications as discussed herein.

Data Set Repository. The training data set repository 612 includes acquired, labeled biophysical data sets (e.g., for cardiac, photoplethysmographic, ballistocardiographic, signal data sets, among other modality data sets discussed herein) acquired from a plurality of clinical studies to be used for the assessment of a given clinical application. For example, for the clinical evaluation system (e.g., 103) used to assess for elevated PCWP, the training data set repository 612 can include cardiac and photoplethysmographic signals.

The training data set repository 1108 may be used to store labeled data sets for other clinical applications as discussed herein.

Univariate Feature Selection module. The univariate feature selection module 612 is configured to assess the available features (e.g., from modules 122′ and/or 608) having greater diagnostic utility than their counterparts, e.g., to reduce feature overfitting to the training data that can lead to poor generalization of the model when it is applied to new unseen data. Development system 104 may employ the univariate feature selection module 612 and the feature importance permutation module 616 to select a subset of optimal features to be used in the model development, e.g., performed by the model development module 610. Features that pass either the first and/or both assessment stages (e.g., via modules 612, 616) may be used in the model development module 610. Univariate feature selection modules 612 may be used for various clinical applications discussed herein, including for (i) heart failure (e.g., left-side or right-side heart failure; heart failure with preserved ejection fraction (HFpEF)), (ii) coronary artery disease (CAD), (iii) various forms of pulmonary hypertension (PH) including without limitation pulmonary arterial hypertension (PAH), (iv) abnormal left ventricular ejection fraction (LVEF), and various other diseases or conditions.

Univariate feature selection module 612 can evaluate many scenarios, each defined by a negative and a positive dataset pair using t-test, mutual information, and AUC-ROC evaluation. The t-test is a statistical test that can determine if there is a difference between two sample means from two populations with unknown variances. Here, the t-tests were conducted against a null hypothesis that there is no difference between the means of the feature in these groups, e.g., normal PCWP vs. elevated (for PCWP algorithm development); CAD− vs. CAD+ (for CAD algorithm development). A small p-value (e.g., ≤0.05) indicates strong evidence against the null hypothesis. Mutual information (MI) operations were conducted to assess the dependence of elevated or abnormal PCWP or significant coronary artery disease on certain features.

An MI score greater than one indicates a higher dependency between the variables being evaluated. An MI score less than one indicates a lower dependency of such variables, and an MI score of zero indicates no such dependency.

A receiver operating characteristic curve, or ROC curve, illustrates the diagnostic ability of a binary classifier system as its discrimination threshold is varied. The ROC curve may be created by plotting the true positive rate (TPR) against the false positive rate (FPR) at various threshold settings. AUC-ROC quantifies the area under a receiver operating characteristic (ROC) curve—the larger this area, the more diagnostically useful the model is. The ROC and AUC-ROC value is considered statistically significant when the bottom end of the 95% confidence interval is greater than 0.50.

Table 5 shows an example list of the negative and a positive dataset pair used in the univariate feature selection assessments. Specifically, Table 5 shows positive datasets being defined as having a PCWP measurement greater than 18 mmHg, and negative datasets were defined as having a PCWP measurement less than 18 mmHg or belonging to a patient group determined to have normal PCWP readings.

TABLE 5

| Scenario Number | Negative Dataset | Positive Dataset |
|---|---|---|
| 1 | <18 mmHg | ³18 mmHg |

In the example of the assessment for elevated PCWP, as a clinical application, results from the univariate feature selection module 616 are provided in: U.S. Patent Application no. US2023/0071085, entitled “Methods and Systems for Engineering Visual Features From Biophysical Signals for Use in Characterizing Physiological Systems”; U.S. Patent Application no. US2023/0075570A1, entitled “Methods and Systems for Engineering Power Spectral Features From Biophysical Signals for Use in Characterizing Physiological Systems”; U.S. Patent Application no. US2023/0127355A1, entitled “Method and System for Engineering Respiration Rate-Related Features_From Biophysical Signals for Use in Characterizing Physiological Systems”; U.S. Patent Application no. US2023-0072281A1, entitled “Methods and Systems for Engineering Wavelet-Based Features From Biophysical Signals for Use in Characterizing Physiological Systems”; U.S. Pat. No. 12,324,683, titled “Method and System to Assess Disease Using Cycle Variability Analysis of Cardiac and Photoplethysmographic Signals”; U.S. Patent Application no. US2023/0055617A1, entitled “Methods and Systems for Engineering photoplethysmographic Waveform Features for Use in Characterizing Physiological Systems”; U.S. Patent Application no. US2023/0076069A1, entitled “Methods and Systems for Engineering Cardiac Waveform Features From Biophysical Signals for Use in Characterizing Physiological Systems”; U.S. Patent Application no. US2023/0075634A1, entitled “Methods and Systems for Engineering Conduction Deviation Features From Biophysical Signals for Use in Characterizing Physiological Systems,” each of which is hereby incorporated by reference herein in its entirety. In the example of the assessment for significant coronary arterial disease (as well as PCWP), as another clinical application, results from the univariate feature selection modules 1112 are provided in the U.S. Patent Application no. US2020/0397322-A1, entitled “Method and System to Assess Disease Using Dynamical Analysis of Biophysical Signals”; and U.S. Pat. No. 11,291,379, entitled “Method and System to Assess Disease Using Dynamical Analysis of Cardiac and Photoplethysmographic Signals,” each of which is hereby incorporated by reference herein in its entirety.

For the assessment of significant coronary arterial disease or PCWP, among others, Univariate feature selection module 612 may perform the assessment on paired data between patients having significant lesions in at least two of three major coronary arteries (positive dataset) and patients having no lesions (negative dataset). Examples of other negative data sets that may be used include healthy control patients, patients with negative diagnostic tests (e.g., as determined using computed tomographic angiography) who do not proceed to cardiac catheterization, and patients who are subjected to angiography but were deemed to have no coronary lesions.

Feature Importance Permutation Analysis Module. The feature importance permutation analysis module 616 is configured to perform additional assessments subsequent to model generation to reduce the number of selected features and their associated models. The feature importance permutation analysis module 616 can generate a permutation importance metric that quantifies the utility of the feature in combination with all other features included in the model generation process. The feature importance permutation analysis module 616 can act as a part of a secondary feature assessment stage that can further limit the number of selected features used in the development of some base models to provide further and more rigorous overfitting control to produce a more stable performance on unseen data.

During model generation (e.g., via Module 610 described below), a large set of models may be generated (e.g., 10,000 models) using a portion of the training data (e.g., 80% of the training data by random sampling) and the rest used for testing. After the training operation, the Feature importance permutation analysis module 616 can assess the features within each model using a feature-specific measure relating to permutation importance (or sensitivity). Permutation importance may be applied to Random Forests and various other classifiers and machine learning models described herein. The feature importance permutation analysis module 616 can permutate the value of each feature such that each patient's feature value is assigned to a different patient to assess whether the feature is important in rendering the model estimation by determining if a given permutation operation causes a significant detriment to the estimation performance. A feature that causes greater detriment to the model performance when permuted as compared to another feature, thus has greater permutation importance.

Intuitively, the permutation importance metric can assess the value that each feature contributes to a model. In some implementations, for a given model, Feature importance permutation analysis module 616 can first compute an initial estimation of the patient's score (e.g., 118) and AUC. The feature importance permutation analysis module 616 can then randomly permutate the values of only a specific feature to recompute the estimation for the score and the revised AUC. The difference between the AUC before and after applying the permutation is the permutation importance of the selected feature. The process can be repeated for all the models and all the features within the models. Feature importance permutation analysis module 616 can assign each feature a permutation importance value using the average of its permutation importance from models that contained that feature as an input.

Model Development Module. The model development module 610 is configured to perform machine learning operations using selected features and labeled data (i.e., data with known disease status) having passed univariate feature selection (e.g., via module 612. Machine learning operations may include but are not limited to linear models, decision trees, random forests, support vector machine, and neural networks such as Random Forest, XGBoost, CatBoost, and Elastic Net models.

Linear models and their variants (e.g., regularized linear models) generate a linear combination of the input features. Decision trees (e.g., gradient boosted decision tree such as XGBoost classifier) generates a series of decisions based on feature thresholds until the output is determined. Random forests generate a collection of decision trees in which each decision tree is trained on various subsets of the training data and the features. Support vector machines generate linear or non-linear classification by mapping the features into a high-dimensional feature space using a kernel operator and then defining a maximally separating hyperplane in that high-dimensional space. Neural networks generate a group of connected units (inspired by a biological neural network) typically organized into an input layer, a set of hidden layers (that can perform linear and non-linear transformations to the features), and an output layer. The ML algorithm, along with its hyper-parameters, may then be determined per the set of available features and per the problem domain.

In one example, the XGBoost Classifier can be performed with hyperparameters comprising a learning rate of 0.5, maximum depth of 3, minimum child weight of 3, 100 total estimators, and a regularization alpha of 0.1. System 103 may use the XGBoost classifier model, e.g., comprising 162 features generated from multiple test labels, including PCWP £ 12 vs. PCWP³ 20, Healthy control group 1 (comprising younger healthy controls) vs. PCWP³ 20, Healthy control group 2 (comprising older healthy controls) vs. PCWP³ 20.

Once the machine learning models (e.g., classifier models 606) have been generated, Module 610 can perform cross-validation operations to reduce overfitting and ensure a certain level of robustness in the estimation (e.g., of score 118) using the machine learning model.

Cross-validation may be performed in an iterative manner in which data is sampled from the available dataset using stratification that samples the data with appropriate weighting in the sampling to ensure an expected representation from both the disease positive and negative groups until a certain percentage of the data has been chosen for training (e.g., 75%-90%). The remaining data can be reserved for validation. A given classifier model is configured and/or optimized using a training data set and then tested on a validation data set. The process may be repeated X times (e.g., 100 times to capture the variability of test performance), resulting in X performance values from which an average performance and variation (such as standard deviation or 95% confidence intervals) can be calculated.

In some embodiments, stratified k-fold cross-validation is performed in which the data is divided while preserving class ratios into k groups or folds. One-fold may be kept for testing, and the model is trained on remaining data (k−1 folds), and the performance metric is evaluated on the testing fold. The process may be repeated k times in which each time for a different fold used for testing and average performance is computed by taking the mean performance of the k folds. The result may be a robust estimate of the performance as it eliminates the errors introduced by random chance. Examples of other cross-validation operations that may be used include Y. Zhang et al., “Facial Emotion Recognition Based on Biorthogonal Wavelet Entropy, Fuzzy Support Vector Machine, and Stratified Cross Validation,” in IEEE Access, vol. 4, pp. 8375-8385, 2016; and Mancuso et al., “Sequential testing in high stakes OSCE: a stratified cross-validation approach,” MedEdPublish, 8, 62 (2019).

FIG. 7A shows an example 10-fold cross-validation setup in which the ML algorithm is trained ten times (702) using a different subset of input data (non-highlighted segment in each row) (704). The performance metric may be calculated for each of these ten trained models using the test data (highlighted block in each row). For example, in the example shown in FIG. 7A, the first model 706 (training #1) is generated using training data subset #2 to #10, and the second model 708 (training #2) is generated using training data subset #1 and #3 to #10, and so forth. The average performance of these ten models on each of their respective test data is reported as the model performance.

To further increase the robustness, variability of the expected performance may be reduced by performing multiple iterations of the cross-validation, e.g., shown in FIG. 7A, with different folds from the same data. The results are combined from the multiple rounds to derive tighter bounds of expected performance. Examples of a cross-validation software framework (CV Framework) that may be used include the open-source ML library sci-kit-learn as described in Pedregosa, Fabian, et al. “Scikit-learn: Machine learning in Python.” The Journal of Machine Learning Research 12 (2011): 2825-2830. The training of models may be performed on various training sets with different characteristics.

The multiple training operations can generate a plurality of models that can be combined in an ensemble operation. Combining models in the ensemble operation can improve the balance between underfitting and overfitting. In underfitting, a model cannot sufficiently capture the complexity of the relationship between the features and label in the training data, resulting in poor performance in both the training data and unseen test data. In overfitting, a model has leveraged spurious relationships between the features and labels in the training data, causing high performance on the training data that drops when those expected spurious relationships are not present in unseen data. Combining models using ensemble techniques allows these two effects to be balanced by either attempting to reduce overfitting when constituent models have higher performance on the training data or attempting to reduce underfitting when constituent models have lower performance on training data with the overall goal of maximizing performance on unseen data.

In the example for PCWP assessment, Model development module 610 generated 13 sets of ML models using the ML algorithms described herein. For the ensemble operation, Model development module 610 can perform the operation as disclosed in relation to the description of FIG. 1. Analytical engine or analyzer (e.g., 806, 814—see FIG. 8) can also average the model estimation with a sigmoid function of the patient BMI having the form

$$( sigmoid ( x ) = \frac{1}{1 + {e}^{- x}} ) .$$

Hyperparameter Sensitivity Module. Hyperparameter sensitivity analysis module 614 is configured to select a subset of the model, e.g., generated from machine learning (ML) experiments (e.g., among a set of 10-20). An ML Experiment can be defined as a single selection for each feature assessment, dataset, and classifier. Within an ML experiment, ML models can be generated using potentially hundreds of hyperparameters and options. In the example of elevated PCWP assessment, Hyperparameter sensitivity analysis module 614 conducted 13 ML experiments based on their average performance across about 100 hyperparameter options, including cross-validation, test performance, and performance on the complete dataset and healthy controls.

Hyperparameter sensitivity analysis module 614 can analyze the sensitivity of the model in the sets of ML experiments to reduce the set down to a single model for each experiment. Hyperparameter sensitivity analysis module 614 can calculate a “hyperparameter sensitivity score” to identify hyperparameters with very little effect on the performance (i.e., changing their values does not affect performance) and hyperparameters that are influential. Hyperparameter sensitivity analysis module 614 can first assess the effect on specific regions of hyperparameters, e.g., where certain values of the hyperparameter may result in very similar performances, but other values can cause the performance to jump or fall. Hyperparameter sensitivity analysis module 614 can then assess the models with the hyperparameters having the higher hyperparameter sensitivity score (e.g., 3-5 hyperparameters from an initial 100's).

The hyperparameter sensitivity score can be used to quantify the “stability” of a given model's hyperparameters—e.g., the degree to which the performance of the model can change when compared to the direct neighbors of a model. Hyperparameter sensitivity analysis module 614 can change one hyperparameter at a time in single discrete steps. If the hyperparameter sensitivity score value is close to zero, then the hyperparameters can be considered robust since a small perturbation was observed to not cause a significant change in performance (a desirable property). In the example shown in FIG. 7B, a score (710) for a model (having a value of 0.67) is compared to the scores of its neighbors (712, 714, 716, and 718) (having values of 0.64, 0.68, 0.65, and 0.66). The scores are generated in FIG. 7B for two hyperparameters “A” and “B” that are varied among “1”, “2”, “3” and among “0.1”, “0.5”, “0.9,” respectively. The difference between the score 710 and the average of the neighbors (712, 714, 716, and 718) in this example is 0.01, which indicates that the hyperparameter and its values (shown as 720 and 722) used to generate the model score (710) are relatively stable and can provide slightly higher performance as compared to its neighboring parameters and their associated values (724).

### Example Clinical and Diagnostic System

FIG. 8 shows an example clinical evaluation system 103 (shown as 103′) that implements the modules of FIG. 1 to non-invasively estimate one or more metrics associated with the physiological state of a patient according to an embodiment. Indeed, the feature modules (e.g., of FIG. 1) can be generally viewed as a part of a system (e.g., the clinical evaluation system 103′) in which any number and/or types of features may be utilized for a disease state, medical condition, an indication of either, or combination thereof that is of interest, e.g., with different embodiments having different configurations of feature modules. FIG. 8 further illustrates the clinical evaluation system 800 as a modular system in which disease-specific add-on modules 802 (e.g., to assess for elevated PCWP or mPAP, CAD, PH/PAH, abnormal LVEF, HFpEF, and others described herein) are capable of being integrated alone or in multiple instances with a singular platform (i.e., a base system 804) to realize system's (e.g., 103′) full operation. The modularity allows the clinical evaluation system 103′ to be designed to leverage the same synchronously acquired biophysical signals and data set and base platform to assess for the presence of several different diseases as such disease-specific algorithms are developed, thereby reducing testing and certification time and cost.

In various embodiments, as shown in FIGS. 1 and 8, different versions of the clinical evaluation system (e.g., 103, 103′) may be implemented by having included different feature computation modules (e.g., 132, 132′) that can be configured for a given disease state(s), medical condition(s), or indicating condition(s) of interest. In another embodiment, the clinical evaluation system (e.g., 103, 103′) may include more than one such system and may be selectively utilized to generate different scores specific to a classifier (shown as 134, 134′) of that engine (e.g., 103, 103′). In this way, the modules of FIGS. 1 and 8 in a more general sense may be viewed as one configuration of a modular system in which different and/or multiple engines (e.g., 103, 103′), with different and/or multiple corresponding classifiers (e.g., 134, 134′), may be used depending on the configuration of module desired.

In the example shown in FIG. 8, Clinical evaluation system 103′ can analyze one or more biophysical-signal data sets (e.g., 110 in FIG. 1) from repository 112 (shown as 112′) using machine-learned disease-specific algorithms to assess the likelihood of a disease, condition, or indication of either, such as elevated PCWP as one example. Clinical evaluation system 103′ includes hardware and software components that are designed to work together in combination to facilitate the analysis and presentation of an estimation score using the algorithm to allow a physician to use that score, e.g., to assess for the presence or non-presence of a disease state, medical condition, or an indication of either.

The base system 804 can provide a foundation of functions and instructions upon which each add-on module 802 (which includes the disease-specific algorithm) then interfaces to assess for the pathology or indicating condition. The base system 804, as shown in the example of FIG. 8, includes a base analytical engine or analyzer 806, a web-service data transfer API 808 (shown as “DTAPI” 808), a report database 810, a web portal service module 812 (e.g., implementing the physician portal visualization 136), and the data repository 112′.

Data repository 112′, which can be cloud-based, stores data from the signal capture system 102 (shown as 102′). Biophysical signal capture system 102′ can be a reusable device designed as a single unit, e.g., with a seven-channel lead set for cardiac signal acquisition and photoplethysmogram (PPG) sensor securely attached (i.e., not removable). Signal capture system 102′, together with its hardware, firmware, and software, provides a user interface to collect patient-specific metadata entered therein (e.g., name, gender, date of birth, medical record number, height, and weight, etc.) to synchronously acquire the patient's electrical and hemodynamic signals. The signal capture system 102′ may securely transmit the metadata and signal data as a single data package directly to the cloud-based data repository. The data repository 112′, in some embodiments, is a secure cloud-based database configured to accept and store the patient-specific data package and allow for its retrieval by the analytical engines or analyzer 806 or 814.

Base analytical engine or analyzer 806 can be a secure cloud-based processing tool that may perform quality assessments of the acquired signals (e.g., performed via “SQA” module 130 (shown as 130′)), the results of which can be communicated to the physician or patient at the point of care. The base analytical engine or analyzer 806 may also perform pre-processing (shown via pre-processing module 818) of the acquired biophysical signals (e.g., 110—see FIG. 1). Web portal 812 is a secure web-based portal configured to provide healthcare providers access to the patient's reports. An example output of the web portal 812 is shown by visualization 136 (also referenced as 402) (see FIGS. 1 and 4). The report databases 810 is a secure database and may securely interface and communicate with other systems, such as a hospital or physician-hosted, remotely hosted, or remote electronic health records systems (e.g., Epic, Cerner, Allscrips, CureMD, Kareo, etc.) so that output score(s) (e.g., 118) and related information may be integrated into and saved with the patient's general health record. In some embodiments, web portal 812 is accessed by a call center to provide the output clinical information over a telephone. Report database 810 may be accessed by other systems that can generate a report to be delivered via the mail, courier service, personal delivery, etc.

Add-on module 802 includes a second part 814 (also referred to herein as the analytical engine (AE) or analyzer 814 and shown as “AE add-on module” 814) that operates with the base analytical engine (AE) or analyzer 806. Analytical engine (AE) or analyzer 814 can include the main function loop of a given disease-specific algorithm, e.g., the feature computation module 132 (shown as 132′), the classifier model 134 (shown as “Ensemble” 134′), and the outlier assessment and rejection module 138 (shown as “Outlier Detection” 138′). In certain modular configurations, Analytical engines or analyzers (e.g., 806 and 814) may be implemented in a single analytical engine module.

The main function loop can include instructions to (i) validate the executing environment to ensure all required environment variables values are present and (ii) execute an analysis pipeline that analyzes a new signal capture data file comprising the acquired biophysical signals to calculate the patient's score using the disease-specific algorithm. To execute the analysis pipeline, AE add-on module 814 can include and execute instructions for the feature modules 132′ and classifier module 134′ to determine an output score (e.g., 118—see FIG. 1 or 4) of the metrics associated with the physiological state of a patient. The analysis pipeline in the AE add-on module 814 can compute the features or parameters (via module(s) 132′) and can identify whether the computed features are outliers (e.g., via modules 138′) by providing an outlier detection return comprising a signal-level response of outlier vs. non-outlier based on the feature. The outliers may be assessed with respect to the training data set used to establish the classifier. AE add-on module 814 can generate the patient's output score (e.g., 118) (e.g., via classier module 134′) using the computed values of the features and classifier models. In the example of an evaluation algorithm for the estimation of elevated PCWP, the output score (e.g., 118) is an PCWP score.

Clinical evaluation system 800 can manage data within and across components using the web-service DTAPIs 808 (also referred to as HCPP web services in some embodiments). DTAPIs 808 may be used to retrieve acquired biophysical data sets from and to store signal quality analysis results to the data repository 112a. DTAPIs 808 may also be invoked to retrieve and provide the stored biophysical data files to the analytical engines or analyzers (e.g., 806, 814). The results of the analytical engine's analysis of the patient signals may be transferred using DTAPI 808 to the report database 810. DTAPIs 808 may also be used, upon a request by a healthcare professional, to retrieve a given patient data set to the web portal module 812, which may present a report to the healthcare practitioner for review and interpretation in a secure web-accessible interface.

Clinical evaluation system 800 can include one or more feature libraries 608, or portions thereof (shown as 608′) that store the feature modules 132′. The feature libraries 608′ may be a part of the add-on modules 802 (as shown in FIG. 8) or the base system 804 (not shown) and are accessed, in some embodiments, by the AE add-on module 814.

Further details of the modularity of modules and various configurations are provided in a provisional patent application currently filed herewith and having attorney docket no. 10321-060pv1, which is hereby incorporated by reference herein in its entirety.

While the methods and systems have been described in connection with certain embodiments and specific examples, it is not intended that the scope be limited to the particular embodiments set forth, as the embodiments herein are intended in all respects to be illustrative rather than restrictive. The clinical evaluation system and method discussed herein may be employed to make, or to assist a physician or other healthcare provider in making, noninvasive diagnoses or determinations of the presence or non-presence and/or severity of other diseases and/or conditions, such as, e.g., coronary artery disease, pulmonary hypertension and other pathologies as described herein using similar or other development approaches. In addition, the example clinical evaluation system and method can be used in the diagnosis and treatment of other cardiac-related pathologies and conditions as well as neurological-related pathologies and conditions, such assessment can be applied to the diagnosis and treatment (including surgical, minimally invasive, and/or pharmacologic treatment) of any pathologies or conditions in which a biophysical signal is involved in any relevant system of a living body. One example in the cardiac context is the diagnosis of CAD and other diseases and conditions disclosed herein and its treatment by any number of therapies, alone or in combination, such as the placement of a stent in a coronary artery, the performance of an atherectomy, angioplasty, prescription of drug therapy, and/or the prescription of exercise, nutritional and other lifestyle changes, etc. Other cardiac-related pathologies or conditions that may be diagnosed include, e.g., arrhythmia, congestive heart failure, valve failure, pulmonary hypertension (e.g., pulmonary arterial hypertension, pulmonary hypertension due to left heart disease, pulmonary hypertension due to lung disease, pulmonary hypertension due to chronic blood clots, and pulmonary hypertension due to other diseases such as blood or other disorders), as well as other cardiac-related pathologies, conditions and/or diseases. Non-limiting examples of neurological-related diseases, pathologies or conditions that may be diagnosed include, e.g., epilepsy, schizophrenia, Parkinson's Disease, Alzheimer's Disease (and all other forms of dementia), autism spectrum (including Asperger syndrome), attention deficit hyperactivity disorder, Huntington's Disease, muscular dystrophy, depression, bipolar disorder, brain/spinal cord tumors (malignant and benign), movement disorders, cognitive impairment, speech impairment, various psychoses, brain/spinal cord/nerve injury, chronic traumatic encephalopathy, cluster headaches, migraine headaches, neuropathy (in its various forms, including peripheral neuropathy), phantom limb/pain, chronic fatigue syndrome, acute and/or chronic pain (including back pain, failed back surgery syndrome, etc.), dyskinesia, anxiety disorders, conditions caused by infections or foreign agents (e.g., Lyme disease, encephalitis, rabies), narcolepsy and other sleep disorders, post-traumatic stress disorder, neurological conditions/effects related to stroke, aneurysms, hemorrhagic injury, etc., tinnitus and other hearing-related diseases/conditions and vision-related diseases/conditions.

In addition, the clinical evaluation system described herein may be configured to analyze biophysical signals such as an electrocardiogram (ECG), electroencephalogram (EEG), gamma synchrony, respiratory function signals, pulse oximetry signals, perfusion data signals; quasi-periodic biological signals, fetal ECG signals, blood pressure signals; cardiac magnetic field signals, heart rate signals, among others.

Further examples of processing that may be used with the exemplified method and system disclosed herein are described in: U.S. Pat. Nos. 9,289,150; 9,655,536; 9,968,275; 8,923,958; 9,408,543; 9,955,883; 9,737,229; 10,039,468; 9,597,021; 9,968,265; 9,910,964; 10,672,518; 10,566,091; 10,566,092; 10,542,897; 10,362,950; 10,292,596; 10,806,349; U.S. Patent Publication nos. 2020/0335217; 2020/0229724; 2019/0214137; 2018/0249960; 2019/0200893; 2019/0384757; 2020/0211713; 2019/0365265; 2020/0205739; 2020/0205745; 2019/0026430; 2019/0026431; PCT Publication nos. WO2017/033164; WO2017/221221; WO2019/130272; WO2018/158749; WO2019/077414; WO2019/130273; WO2019/244043; WO2020/136569; WO2019/234587; WO2020/136570; WO2020/136571; U.S. patent application Ser. Nos. 16/831,264; 16/831,380; 17/132,869; PCT Application nos. PCT/IB2020/052889; PCT/IB2020/052890, each of which has been incorporated by reference herein in its entirety.

The following patents, applications, and publications as listed below and throughout this document are hereby incorporated by reference in their entirety herein.

### LIST OF REFERENCES

[1] Y. Zhang et al., “Facial Emotion Recognition Based on Biorthogonal Wavelet Entropy, Fuzzy Support Vector Machine, and Stratified Cross Validation,” in IEEE Access, vol. 4, pp. 8375-8385, 2016, doi: 10.1109/ACCESS.2016.2628407.
[2] Mancuso G, Strachan S, Capey S, 2019, ‘Sequential testing in high stakes OSCE: a stratified cross-validation approach’, MedEdPublish, 8, [2], 62, https://doi.org/10.15694/mep.2019. 000132.1
[3] Pedregosa, Fabian, et al. “Scikit-learn: Machine learning in Python.” The Journal of Machine Learning Research 12 (2011): 2825-2830.
[4] Breiman, “Random Forests,” Machine Learning, 45(1), 5-32, 2001.
[5] Chen, Tianqi; Guestrin, Carlos (2016). “XGBoost: A Scalable Tree Boosting System,” Proceedings of the 22nd ACM SIGKDD International Conference on Knowledge Discovery and Data Mining, San Francisco, CA, USA, Aug. 13-17, 2016. ACM. pp. 785-794.
[6] H. Zou and T. Hastie, “Regularization and variable selection via the elastic net,” Journal of the Roy. Statist. Soc., ser. B, vol. 67, no. 2, pp. 301-320, 2005.
[7] L. Breiman, “Bagging predictors,” Machine Learning, 24(2), 123-140, 1996.
[8] Liu, F. T., Ting, K. M. and Zhou, Z. H., 2008 December. Isolation forest. In 2008 eighth IEEE international conference on data mining (pp. 413-422). IEEE.
[9] Oh, Jae K. et al. “Diagnosis of Heart Failure With Preserved Ejection Fraction Relies on Detection of Increased Diastolic Filling Pressure, But How?” J Am Heart Assoc. 12, 2023. DOI: 10.1161/JAHA.122.028867.
[10] von Haehling, Stephen et al. “Heart failure with preserved ejection fraction: diagnosis, risk assessment, and treatment,” Clinical Research in Cardiology (2024) 113:1287-1305.
[11] Wattanachayakul, Phuuwadith, et al. “Invasive haemodynamic assessment in heart failure with preserved ejection fraction,” ESC Heart Failure 2025; 12:1558-1570.
[12] Borlaug, B. A., & Redfield, M. M. (2011). Diastolic and systolic heart failure are distinct phenotypes within the heart failure spectrum. Circulation, 123(18), 2006-2014.
[13] Paulus, W. J., & Tschope, C. (2013). A novel paradigm for heart failure with preserved ejection fraction: comorbidities drive myocardial dysfunction and remodeling through coronary microvascular endothelial inflammation. Journal of the American College of Cardiology, 62(4), 263-271.
[14] Ware, L. B., & Matthay, M. A. (2000). The acute respiratory distress syndrome. The New England journal of medicine, 342(18), 1334-1349.
[15] Humbert, M., Kovacs, G., Hoeper, M. M., Badagliacca, R., Berger, R. M. F., Brida, M., . . . Ferrari, P. (2022). 2022 ESC/ERS Guidelines for the diagnosis and treatment of pulmonary hypertension: Developed by the task force for the diagnosis and treatment of pulmonary hypertension of the European Society of Cardiology (ESC) and the European Respiratory Society (ERS). Endorsed by the International Society for Heart and Lung Transplantation (ISHLT) and the European Reference Network on rare respiratory diseases (ERN-LUNG). European Heart Journal, 43(38), 3618-3731.
[16] Gerges, M., Gerges, C., & Lang, I. M. (2016). How to define pulmonary hypertension due to left heart disease. The European respiratory journal, 48(2), 553-555.
[17] Anker, S. D., Butler, J., Filippatos, G., Ferreira, J. P., Bocchi, E., Böhm, M., Brunner-La Rocca, H. P., Choi, D., J., Chopra, V., Chuquiure-Valenzuela, E., Giannetti, N., Gomez-Mesa, J. E., Janssens, S., Januzzi, J. L., Gonzalez-Juanatey, J. R., Merkely, B., Nicholls, S. J., Perrone, S. V., Piña, I. L., Ponikowski, P., . . . EMPEROR-Preserved Trial Investigators (2021). Empagliflozin in Heart Failure with a Preserved Ejection Fraction. The New England journal of medicine, 385(16), 1451-1461.
[18] Cury, R. C., Abbara, S., Achenbach, S., Agatston, A., Berman, D. S., Budoff, M. J., . . . Rubin, G. D. (2016). CAD-RADSTM coronary artery disease-reporting and data system. An expert consensus document of the Society of Cardiovascular Computed Tomography (SCCT), the American College of Radiology (ACR) and the North American Society for Cardiovascular Imaging (NASC. Journal of Cardiovascular Computed Tomography, 10(4), 269-281.
[19] Dorogush, A. V., Ershov, V., & Gulin, A. (2018). CatBoost: gradient boosting with categorical features support. arXiv preprint arXiv:1810.11363.
[20] Prokhorenkova, L., Gusev, G., Vorobev, A., Dorogush, A. V., & Gulin, A. (2018). CatBoost: unbiased boosting with categorical features. Advances in Neural Information Processing Systems, 31.
[21] Oh, Jae K., William R. Miranda, and Garvan C. Kane. “Diagnosis of heart failure with preserved ejection fraction relies on detection of increased diastolic filling pressure, but how?.” Journal of the American Heart Association 12.6 (2023): e028867.
[22] von Haehling, Stephan, et al. “Heart failure with preserved ejection fraction: diagnosis, risk assessment, and treatment.” Clinical Research in Cardiology 113.9 (2024): 1287-1305.
[23] Zile M R, Brutsaert D L (2002) New concepts in diastolic dysfunction and diastolic heart failure: part I: diagnosis, prognosis, and measurements of diastolic function. Circulation 5:1387-1393
[24] Wattanachayakul, Phuuwadith, et al. “Invasive haemodynamic assessment in heart failure with preserved ejection fraction.” ESC Heart Failure 12.3 (2025): 1558-1570.

1. A method to non-invasively estimate the presence of elevated pulmonary capillary wedge pressure (PCWP) in a mammalian subject, the method comprising:
obtaining, by one or more processors, a first biophysical signal data set associated with a first photoplethysmographic signal and a second photoplethysmographic signal, wherein the first biophysical data set has been acquired over multiple cardiac cycles of the subject;
obtaining, by the one or more processors, a second biophysical signal data set associated with a cardiac signal, wherein the second biophysical data set has been simultaneously acquired with the first biophysical signal data set over the multiple cardiac cycles;
determining, by the one or more processors utilizing at least a portion of the first and second biophysical signal data sets, a plurality of values associated with a plurality of features; and
determining, by the one or more processors, an estimated value for the presence of elevated pulmonary capillary wedge pressure,
wherein the estimated value for the presence of abnormal pulmonary capillary wedge pressure is outputted for use in a diagnosis of expected heart failure (HF) or to direct treatment of the expected heart failure.

2. The method of claim 1, wherein the plurality of features include a feature set selected from the group consisting of:
one or more depolarization or repolarization wave propagation associated features;
one or more depolarization wave propagation deviation associated features;
one or more cycle variability-associated features;
one or more dynamical systems associated features;
one or more cardiac waveform topological and variations associated features;
one or more photoplethysmogram (PPG) waveform topologic and variations associated features;
one or more cardiac or PPG signal power spectral density associated features; and
one or more cardiac or PPG signal visual associated features.

3. The method of claim 2, wherein the one or more depolarization or repolarization wave propagation associated features are configured to quantify a propagative characteristic of a ventricular depolarization (VD) wave or a ventricular repolarization (VR) wave defined in the cardiac signal in three-dimensional space.

4. The method of claim 2, wherein the one or more depolarization or repolarization wave propagation associated features are configured to quantify a propagative characteristic of a bandpass filtered ventricular depolarization (VD) wave or a ventricular repolarization (VR) wave defined in the cardiac signal in three-dimensional space.

5. The method of claim 2, wherein the one or more depolarization wave propagation deviation associated features are configured to quantify deviations, via evaluation of high-frequency and low-amplitude patterns, of a VD wave trajectory from a trajectory of a three-dimensional modeled VD wave, wherein the three-dimensional modeled VD wave is a representative VD wave below about 40 Hz.

6. The method of claim 2, wherein the one or more cycle variability associated features are configured to quantify beat-to-beat variations of the cardiac signal in comparison of each respective beat to a determined template beat.

7. The method of claim 2, wherein the one or more dynamical system features are configured to quantify dynamical characteristics of the second biophysical data set.

8. The method of claim 2, wherein the one or more cardiac waveform topologic and variations associated features are configured to quantify both cardiac measurements and variations of the second biophysical signal data set.

9. The method of claim 2, wherein the one or more PPG waveform topologic and variations associated features are configured to quantify both PPG measurements and variations of the first biophysical signal data set.

10. The method of claim 2, wherein the one or more cardiac or PPG signal power spectral density associated features are configured to quantify a power spectrum and frequency content of a first PPG signal and a second PPG signal.

11. The method of claim 2, wherein the one or more cardiac or PPG signal power spectral density associated features are configured to quantify a power spectrum and frequency content of second biophysical signal data set.

12. The method of claim 2, wherein the one or more cardiac or PPG visual associated features are configured to quantify geometric parameters of a three-dimensional phase space generated from the first or second biophysical data set.

13. The method of claim 1, wherein the plurality of features include a feature set selected from the group consisting of:
one or more cardiac or PPG signal wavelet-associated features;
one or more cardiac or PPG signal-associated LVEDP features; and
one or more cardiac or PPG signal associated with linear left ventricular hypertrophy.

14. The method of claim 1, wherein the one or more processors are located in a cloud platform.

15. The method of claim 1, wherein the one or more processors are located in a local computing device.

16. The method of claim 1 further comprising:
determining, by the one or more processors, via an outlier detection analysis of the first biophysical signal data set and the second biophysical signal data set, the presence of outliers, wherein the outlier detection analysis comprises a machine learned module configured to detect arrhythmia and/or incorrect lead configurations in the mammalian subject.

17. The method of claim 16, wherein the machine learned module is further configured to detect an anomalous waveform that is missing or has extra waveform elements in a first PPG signal, a second PPG signal, and/or the cardiac signal.

18. The method of claim 1 further comprising:
preprocessing, by the one or more processors, the second biophysical signal data set, wherein the preprocessing includes i) transient time removal operation, DC offset removal operation, and baseline wander removal operation.

19. The method of claim 1 further comprising:
determining, by the one or more processors, via signal quality assessment analysis of the first biophysical signal data set, a plurality of PPG noise scores of the first biophysical signal data set, including i) a first PPG noise score associated with rapid changes in first or second PPG signals and ii) a second PPG noise score associated with measurement saturation.

20. The method of claim 1 further comprising:
determining, by the one or more processors, via signal quality assessment analysis of the second biophysical signal data set, a plurality of biopotential noise scores of the second biophysical signal data set, including a first biopotential noise score associated with biopotential powerline interference and a second biopotential noise score associated with biopotential high frequency noise.

21. The method of claim 1, wherein the step of determining the estimated value for the presence of elevated pulmonary capillary wedge pressure values is determined by:
determining, by the one or more processors, a plurality of values for the plurality of features in two or more models, including a model selected from the group consisting of a linear model, a decision tree model, a random forest model, a support vector machine model, a neural network model, and wherein the two or more models are combined in an ensemble model that outputs the estimated value for the presence of abnormal pulmonary capillary wedge pressure.

22. The method of claim 21, wherein the ensemble model includes at least the two or more models and a model associated with a body mass index (BMI) value of the mammalian subject.

23. The method of claim 1, wherein the plurality of features are selected from a pool of candidate features by a selection module.

24. The method of claim 23, wherein the selection module comprises a univariate feature assessment analysis that evaluates data sets defined with respect to a positive pathology label associated with an elevated PCWP and a negative pathology label associated with a normal PCWP.

25. The method of claim 24, wherein the data sets are defined by a positive and negative pathology label selected from the group consisting of:
a PCWP value greater than or equal to 18 mmHg and a PCWP value less than or equal to 18 mmHg.

26. The method of claim 1, wherein a univariate feature assessment analysis is configured to determine a receiver-operating characteristic curve, a t-test, or a normalized mutual information analysis for presence of the elevated pulmonary capillary wedge pressure.

27. The method of claim 1, further comprising:
removing a feature from a plurality of candidate features having a high occurrence of extraction errors.

28. The method of claim 23, wherein the selection module comprises a cross-validation analysis for a set of trained models, wherein evaluated data sets are randomly sampled as a training data set and non-selected data sets as a remainder are used as a validation data set in a feature permutation importance analysis.

29. The method of claim 28, wherein the feature permutation importance analysis quantifies a utility of a feature in combination with all other assessed features.

30. The method of claim 23, wherein the plurality of features are configured in a machine-learned model.

31. The method of claim 30, wherein the machine-learned model is trained using hyperparameters selected from a group consisting of hyperparameters that have been evaluated in a hyperparameter sensitivity analysis.

32.-41. (canceled)
